(19) United States
(12) Patent Application Publication
(10) Pub. No.: US 2015/0232576 A1
(43) Pub. Date: Aug. 20, 2015

(54) ENDOTHELIAL NITRIC OXIDE SYNTHASE

(71) Applicants: John C. Salerno, Acworth, GA (US); Carol A. Chrestensen, Marietta, GA (US)

(72) Inventors: John C. Salerno, Acworth, GA (US); Carol A. Chrestensen, Marietta, GA (US)

(73) Assignee: Kennesaw State University Research and Services Foundation, Inc., Kennesaw, GA (US)

(21) Appl. No.: 14/672,642

(22) Filed: Mar. 30, 2015

Related U.S. Application Data
(63) Continuation-in-part of application No. 13/773,721, filed on Feb. 22, 2013.
(60) Provisional application No. 61/637,124, filed on Apr. 23, 2012.
(60) Provisional application No. 61/602,450, filed on Feb. 23, 2012.

(51) Int. Cl. C07K 16/40; G01N 33/573

(52) U.S. Cl. CPC C07K 16/40; G01N 33/573; C07K 2317/24; C07K 2317/622; C07K 2317/55; C07K 2317/54; G01N 2440/14; G01N 2333/90254

(57) ABSTRACT

Endothelial nitric oxide synthase (eNOS) activity was modulated by contact with an effective amount of a mitogen-activated protein (MAP) kinase, resulting in phosphorylation of S602, T46, and/or S58. Kinetics and stoichiometry are disclosed. The contact strongly reduced nitric oxide (NO) synthesis, and inhibited the cytochrome c reductase activity of eNOS reductase domains. Three sites of phosphorylation were determined that matched the serine-proline (SP) and threonine-proline (TP) motifs of typical MAP kinase phosphorylation sites.

### CROSS REFERENCE TO RELATED APPLICATIONS

This application is a Continuation-in-Part of co-pending U.S. patent application Ser. No. 13/773,721 filed Feb. 22, 2013; which claims priority to U.S. Provisional Application Ser. Nos. 61/602,450 filed Feb. 23, 2012, and 61/637,124 filed Apr. 23, 2012, each of which is expressly incorporated by reference herein in its entirety.

This invention was made with government support under NIH 3R15GM080701-01, and NSF 1020261 and 0950920. The government has certain rights in the invention.

Nitric oxide is an important molecular messenger in human physiology. The enzyme endothelial nitric oxide synthase from endothelial cells (eNOS) is a signal integrator in networks for homeostasis, e.g., vascular tone, insulin secretion, angiogenesis, etc. Molecular genetic and biochemical approaches elucidated interactions of purified components and demonstrated that mitogen-activated protein (MAP) kinases regulated eNOS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows alignment of the pentabasic recognition site (bold) in endothelial nitric oxide synthase (eNOS) within the D-site for binding partners for extracellular signal-regulated kinase (ERK), p38 MAP kinase, and c-Jun N-terminal kinase (JNK).

FIGS. 2A-B show binding of eNOS and nNOS to p38 under various conditions, FIG. 2A eNOS and Nnos binding to p38; FIG. 2B sensorgrams of eNOS and nNOS concentration course.

FIGS. 3A-C show binding of eNOS to ERK-2, where FIG. 3A shows the association phase of eNOS and nNOS binding to ERK-2, FIG. 3B shows sensorgrams of eNOS concentration course, and FIG. 3C shows a plot of steady state amplitude against eNOS concentration.

FIG. 4 shows sequence alignment of NOS mammalian isoforms in the autoinhibitory (AI) element region, where the sequences continue from panel A to panel B to panel C, and showing SEQ ID NOS: 6-15.

FIG. 5 shows mass spectroscopy data for trypsin digest of ERK phosphorylated eNOS.

FIGS. 6A-B show structural cartoons of NOS reductase domains (FIG. 6A) and eNOS oxygenase dimer (FIG. 6B).

FIGS. 7A-D show an optical biosensing analysis of CaM-eNOS binding, where FIGS. 7A and 7B show BLI traces with fits to global single-state models for CaM binding to unphosphorylated and ERK phosphorylated eNOS, respectively, and FIGS. 7C and 7D show SPR sensorgram of single-cycle kinetics of tethered CaM binding to unphosphorylated eNOS and phosphorylated eNOS, respectively.

FIG. 8 shows fluorescence decays of unphosphorylated and ERK phosphorylated eNOS holoenzyme FMN in the presence and absence of calmodulin

FIGS. 9A-C show phosphorylation of purified eNOS by ERK2 (FIG. 9A) and time course of phosphorylation (FIG. 9B); FIG. 9C shows loss of signal as phosphate is removed from eNOS by phosphorylase B.

FIGS. 10A-B show phosphorylation of affinity purified eNOS with three MAP kinases, ERK2, p38, and JNK probed with pS116 antibody (FIG. 10A) or the novel pS602 antibody (FIG. 10B).

FIG. 11 is a molecular model of eNOS reductase domains with major control elements extending from N598 to T644 (AI) and from G 1161 to P1203 (C terminus); built on 1TLL.

FIG. 12 is a signaling network associated with the insulin receptor, showing multiple inputs to eNOS.

FIG. 13 shows eNOS phosphorylation in cultured endothelial cells using pS1179 antibody in top A and shows anti-pS602eNOS R2; anti-eNOS; anti-phosAKT 1,2,3; and anti-phos44/42 ERK in bottom B.

FIG. 14 schematically illustrates a feedback network model for oscillation of phosphorylation state in response to external signals; a represents a kinase arm, b a phosphatase arm, and c a passive target.

FIG. 15 is a Runge-Kutta simulation of phosphatase-kinase feedback loop.

The enzyme endothelial nitric oxide synthase (eNOS) is an important signal generator involved in control of vascular and airway tone, insulin secretion, cardiac function, and angiogenesis (6-10). eNOS-generated nitric oxide (NO) is involved in processes such as angiogenesis, ischemia-reperfusion, and insulin response. eNOS is primarily controlled through calcium/calmodulin activation

The eNOS protein contains a motif that is similar to recognition sequences in known mitogen-activated protein (MAP) kinase binding partners that is located within a major eNOS control element. The mitogen-activated protein kinases, specifically extracellular signal regulated kinase (ERK) and p38, when purified, phosphorylated eNOS; the apparent stoichiometry was 2-3 phosphates per eNOS monomer. Phosphorylated eNOS strongly reduced nitric oxide (NO) synthesis and inhibited the cytochrome c reductase activity of the eNOS reductase domains. Phosphorylation of eNOS occurred at three sites, as detected by mass spectroscopy using tryptic digests; each of the three sites matched the serine-proline (SP) and threonine-proline (TP) motifs of typical MAP kinase phosphorylation sites. eNOS bound p38 and ERK with about 100 nM affinity and complex kinetics, as determined by optical biosensing. Binding was diffusion-limited (kₒₙ about 0.15×10⁶ M⁻¹ sec⁻¹). Neuronal NOS also bound p38 but exhibited much slower and weaker binding. p38-eNOS binding was inhibited by calmodulin (CaM). Evidence for a ternary complex was found when eNOS-bound p38 was exposed to CaM, increasing the apparent dissociation rate. These data strongly suggested that MAP kinases directly regulate NOS. Such direct regulation has implications for signaling pathways, including angiogenesis and control of vascular tone.

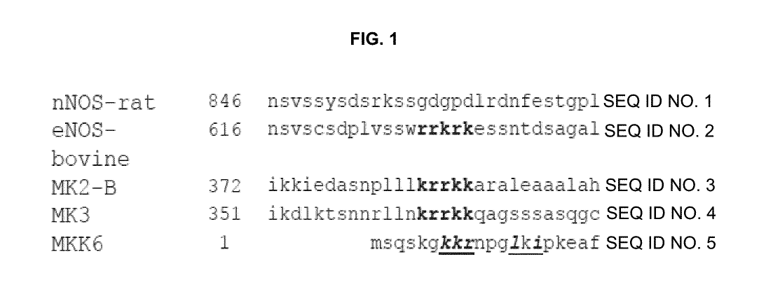

Mitogen activated protein kinases are central nodes in stress-activated signaling networks. MAP kinases regulate gene expression in response to environmental changes, inflammatory cytokines, and other signals. Endothelial nitric oxide synthase (mitogen activated protein kinase 1 and 2 (ERK1/2), p38, and eNOS-generated nitric oxide (NO) are involved in processes such as angiogenesis, ischemia-reperfusion, and insulin response (1-10). ERK1/2 phosphorylates eNOS in a bradykinin-sensitive ERK1/2-murine thymoma viral oncogene homolog 1 (a.k.a, protein kinase B, AKT)-eNOS-Raf-1 complex in bovine aortic endothelial cells (BAECs) (11). p38 is stimulated by upstream kinases from several pathways and activates a broad spectrum of downstream kinases, including MAPKAP kinases 2 and 3 and numerous transcription factors such as activating transcription factor (ATF) 1/2 and myocyte enhancer factor (MEF)2A (3,12). Many binding partners for ERK, p38, and JNK contain a basic recognition site within the D-site (FIG. 1) that facilitates interaction between MAP kinases and other signaling components (1-4, 12). FIG. 1 shows alignment of the pentabasic sequence (bold) in eNOS and the aligned region in neuronal nitric oxide synthase (nNOS) and with the D sites of MK2-B, MK3, and MKK6. The classic D site sequence is characterized by (K/R2-3-X1-6-φ-X-φ) (4), and is bolded and underlined in the MKK6 sequence. MK2-B and MK3 lack the hydrophobic portion of the D site, similar to eNOS. All sequences are human unless otherwise designated; the sequence corresponding to the recognition region is essentially invariant within each group of mammalian kinase and NOS orthologs.

The canonical binding site for recognition by MAP kinases is KKRxxxLxI. Considerable variation from this motif has been postulated to facilitate recognition by a helix and neighboring regions on MAPKs that bear an array of acidic and hydrophobic residues. Among several alternatives, Enslena et al proposed that (R/K)₂-(x)₂₋₆-(L/I)×(L/1) is a more general motif (13). Sharrocks et al presented an even more general scheme for recognition of substrates by MAP kinases involving a multibasic region, an L×L region, and flanking C and N terminal hydrophobic residues; at least two of these four determinants were present in all sequences considered (14). A series of peptides based on putative recognition sites has recently been used to probe the specificity of a variety of MAP kinases. Variations within the D site contribute to, but do not completely account for, differential binding of related substrates to JNK, ERK and p38 type kinases, all of which recognize these motifs (15).

As stated, eNOS is an important signal generator involved in control of vascular tone, insulin secretion, and angiogenesis. eNOS is primarily controlled by displacing an autoinhibitory element by calcium/calmodulin (Ca⁺²/CaM) (16), but eNOS is also regulated by several kinases acting at different sites to activate or inhibit NO production (17-19). eNOS regulation also involves targeting to different compartments by myristolation, palmitoylation, and protein-protein interactions. Some of the activating phosphorylation sites are within the autoinhibitory element (S615 and S633 in human eNOS; S617 and S635 in bovine eNOS), and are phosphorylated by protein kinase A (PKA) and AKT.

The autoinhibitory element contains a conserved region common to CaM regulated NOS enzymes; this includes one of the AKT/PKA (S617 in eNOS) phosphorylation sites and a helical region that forms hydrogen bonds with the FMN binding domain and with the two domain dehydrogenase unit with which it is associated in the available crystal structure (20). nNOS lacks the equivalents of the S602 and S633 sites. The C terminal half of the autoinhibitory element is not conserved at the protein level because of local frame shifts which generated a unique pentabasic sequence in mammalian eNOS. As shown in FIG. 1, the corresponding region of nNOS contains only two basic residues, although there are other basic residues, notably a triplet nearby in the canonical calmodulin-binding motif.

Biosensor experiments provide a powerful and versatile probe of protein-protein interactions, allowing verification of predicted binding and quantitative description of affinity and kinetics of binding and release. The inventors utilized optical biosensing to elucidate calcium-dependent changes to the rates and affinity of calmodulin binding to NOS (21).

The inventors herein disclose that very compact versions of the D site can be detected and bind with high affinity to MAP kinases such as ERK1/2 and p38. ERK1/2 bound to eNOS but did not bind to nNOS. p38 strongly bound to eNOS, but made a much weaker complex with nNOS, consistent with recognition of a pentabasic sequence in eNOS by p38. p38-eNOS binding was inhibited and release was stimulated by CaM, suggesting a weak ternary complex. p38 inhibition of eNOS is consistent with partial competition with CaM.

Signaling networks, including elaborate feedback and feedforward mechanisms, are responsible for homeostasis over multiple levels of organization. Familiar signal transduction cascades, where amplification is obtained by sequential phosphorylation/activation, have been augmented by the discovery of multi-component signaling complexes that may include receptors, kinases, scaffolds and adaptors, and non-kinase signal generators.

While eNOS is primarily controlled through calcium/calmodulin activation, numerous other inputs include inhibitory and activating phosphorylation by specific kinases, protein-protein interactions, and cell trafficking. Examples of activating kinases include PKA which phosphorylates S1177 and S633, and murine thymoma viral oncogene homolog 1 (a.k.a, protein kinase B) (AKT), which phosphorylates S1177 and S615. S1177 is located in the C terminal tail, which acts to restrict the rate of heme reduction by the flavin containing domains. S633 and S615 are located in a large autoinhibitory insertion in the FMN binding domain, which is displaced by CaM binding during activation. PKC inhibits eNOS by phosphorylating T495 adjacent to the CaM binding site, which interferes with CaM binding mediated activation. Other phosphorylation sites associated with the oxygenase domain are less well studied.

MAP kinases including the ERK, p38 and JNK families are important signaling nodes in pathways that control metabolism, growth and expression. ERK and p38 both function in signaling pathways that involve eNOS, and good evidence for direct phosphorylation of eNOS by ERK1/2 in BAECs has been obtained. Contradictory reports of the site of ERK phosphorylation have appeared.

The present invention discloses a pentabasic binding site for MAP kinases in the eNOS autoinhibitory element as well as targets of phosphorylation, the effects on activity, and the mechanism by which phosphorylation regulates NO synthesis.

The present invention discloses peptides that bind to a pentabasic binding site for MAP kinases in the eNOS autoinhibitory element and which may inhibit the binding of MAP kinases.

Embodiments disclose methods for modulating nitric oxide synthase activity by contacting a nitric oxide synthase with an effective amount of peptide for a time sufficient to allow binding of the nitric oxide synthase with the peptide, where contacting the nitric oxide synthase with the peptide results in binding of the peptide to a pentabasic sequence in the auto-inhibitory element of the nitric oxide synthase. Binding of the peptide to the nitric oxide synthase results in modulation of nitric oxide synthase activity.

Embodiments disclose methods for modulating nitric oxide synthase activity by contacting a nitric oxide synthase with an effective amount of a MAP kinase, resulting in phosphorylation of at least one amino acid selected from the group of S602, T46, and S58. Such amino acid phosphorylation results in modulation of nitric oxide synthase activity.

The present invention discloses methods for modulating the cytochrome C reductase activity of nitric oxide synthase. A nitric oxide synthase reductase domain is contacted with an effective amount of a MAP kinase, where phosphorylation of at least one amino acid from the nitric oxide synthase reductase domain results in the modulation of cytochrome C reductase activity.

Embodiments of the present invention also disclose pentabasic peptides and variants thereof.

Embodiments of the present invention also disclose antibodies that bind to a pentabasic binding site for MAP kinases in the eNOS autoinhibitory element.

The present invention also discloses antibodies to phosphorylated peptide analogs that may be used to detect eNOS phosphorylation, either by ERK or by other kinases. Phosphorylation sites include, but are not limited to, S602 in bovine eNOS, S600 in human eNOS, T46 in bovine eNOS, T44 in human eNOS, S58 in bovine eNOS, and S56 in human eNOS. These antibodies may be raised using phosphorylated (e.g., by ERK) synthetic peptides with sequences derived from the region around the site. In various embodiments, the described antibodies are present in a suitable buffer.

Embodiments of the invention include peptidomimetic mutant enzymes with D or E substituted for the phosphorylated S and T residues that may mimic constitutively phosphorylated enzymes.

Embodiments of the invention include peptidomimetic null enzymes with another residue, commonly A for the phosphorylated S and T residues, which would provide enzymes unphosphorylated at that site.

Embodiments of the invention includes mutated enzymes as described above combined with phosphor-mimic and -null mutants at other sites (e.g. S617 or T497), as well as genes coding for these enzymes.

Embodiments of the invention include vectors and expression systems containing genes encoding the enzymes described herein. Such vectors and expression systems may be incorporated into expression systems for use in cell or tissue culture or in animals or humans. Such vectors may also be used in gene therapy methods (e.g., ERK phosphorylation nulls for higher NO production in vivo).

NOSs were expressed and purified as known in the art (22-24). Heme and flavin content were estimated spectrophotometrically, and activity assayed by following NADPH consumption at 340 nm (23, 24). eNOS NO synthase activity and cytochrome c reduction were assayed as known in the art (25, 26), the concentration of CaM was reduced to 0.6 μM because of potential competition between CaM and p38. Phosphorylated His-p38 was expressed as known in the art (26) and was purified using Talon™ resin as directed by the manufacturer. ERK-2 was purchased from PROSPEC (NJ). Biolayer interferometry (27) measurements were made on a ForteBio (Menlo Park Calif.) Octet QK biosensor using streptavidin sensors. Assays were performed in 200 μL volumes at 25° C. p38 and ERK were biotinylated as known in the art (21). Biotinylated kinases were loaded onto sensors for 600 s. After establishing baseline response, kinases were exposed to analyte eNOS or nNOS at a range of concentrations. Baseline, association, and dissociation phases were all performed in NOS buffer (10 mM phosphate, pH 7.5, 100 mM NaCl, 10% glycerol, 0.005% surfactant P-20, 10 uM CaCl₂). Association and dissociation were monitored as nm shift. Nonspecific binding of analytes to sensors without ligand was negligible and so was not subtracted.

Binding and release of analyte was simulated using standard kinetics (21). A single first order model generated acceptable simulations for only the first 30 seconds of binding, but good quality fits were obtained using two components with either sequential (A+B⇄C⇄D) or parallel (A+B⇄C; D+B⇄E) models. Sequential binding was modeled at each analyte concentration with pseudo first order rate constants k₁ and k₂ for the forward and reverse reactions of the initial step and k₃ and k₄ for the forward and reverse rate constants of the second step. Binding curves were calculated numerically and forward and reverse rate constants extracted by simulation at all analyte concentrations examined. Parallel model simulation of the forward reactions was done using summed exponentials; the observed pseudo first order rate constant k_{obs} is then the sum of the forward and reverse rates. In both cases the second order rate constant is the product of analyte concentration and pseudo first order forward rate constant k₁.

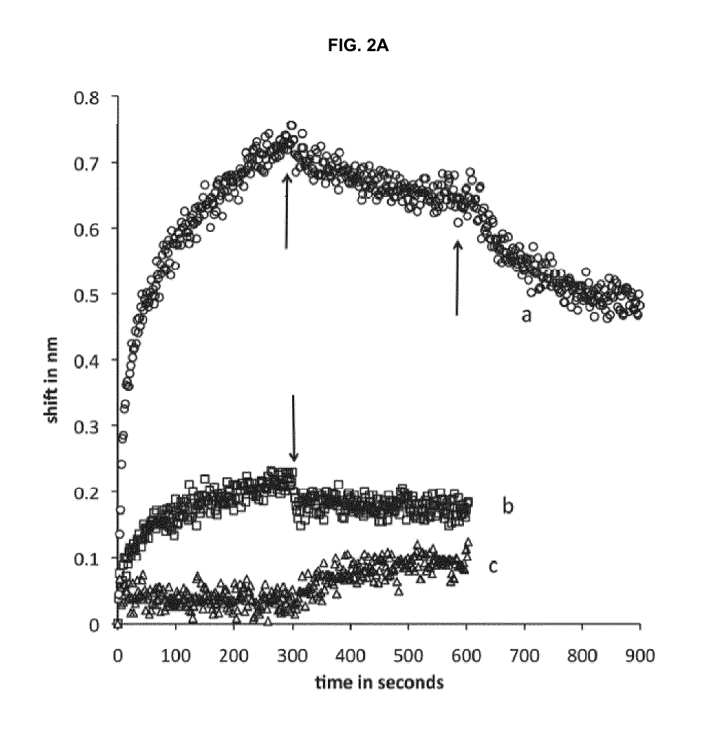

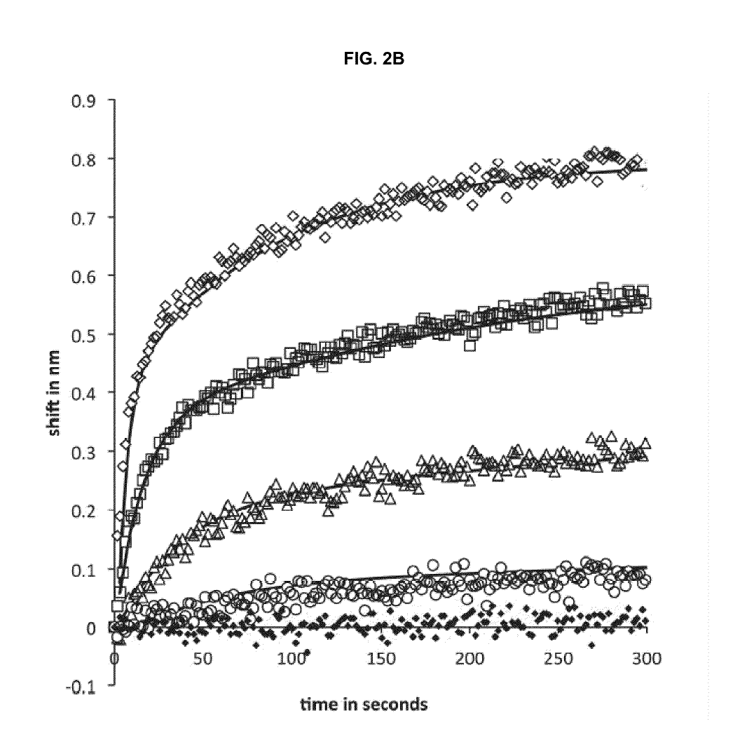

eNOS bound to p38. FIG. 2A trace a (circles) shows binding of eNOS to tethered p38, it shows sensorgrams of eNOS and nNOS binding to p38. Immobilized p38 was immersed in 696 nM NOS at time 0. Binding was measured for 300 s followed by transfer to buffer only and monitoring of dissociation for 300 s. Trace a (circles) was eNOS binding with an additional step in which the tip was moved into buffer with 1 μM CaM after initial dissociation. FIG. 2A trace b (squares) was nNOS binding. FIG. 2A trace c (triangles) was eNOS pre-equilibrated with a fourfold molar excess of CaM prior to immersion of p38. Arrows indicate movement of sensors from association to dissociation or dissociation to CaM-containing buffer.

Binding was rapid; an initial component accounts for about ⅔ of the signal and has a half time of about 10 sec. A second, slower component appears to represent changes in protein conformation on the sensor. After 300 s, the sensor was immersed in buffer only; dissociation of about 25% of the rapid phase binding was observed, consistent with conversion of the remaining complex to a slowly dissociating form in the second phase of association. After dissociation in buffer, the sensor was moved to buffer containing CaM. Dissociation was still incomplete, but was significantly more rapid and extensive. The rapid phase of association was representative of potentially physiologically relevant events; very slow conformational changes of protein associated with the sensor surface likely represented partial unfolding or aggregation.

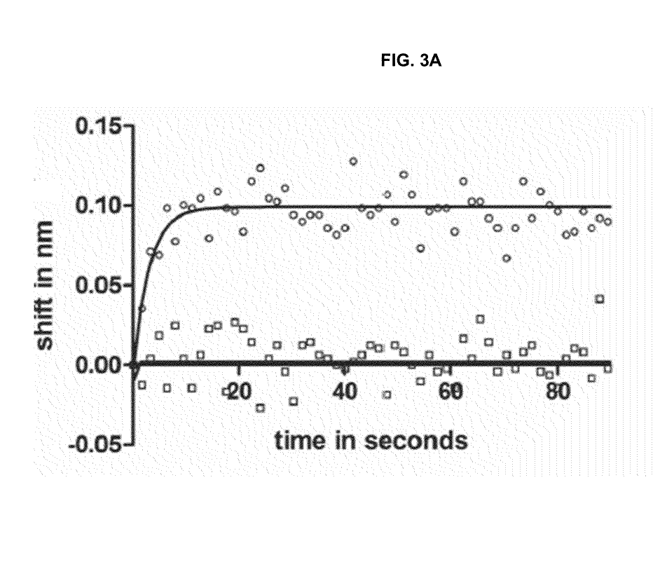

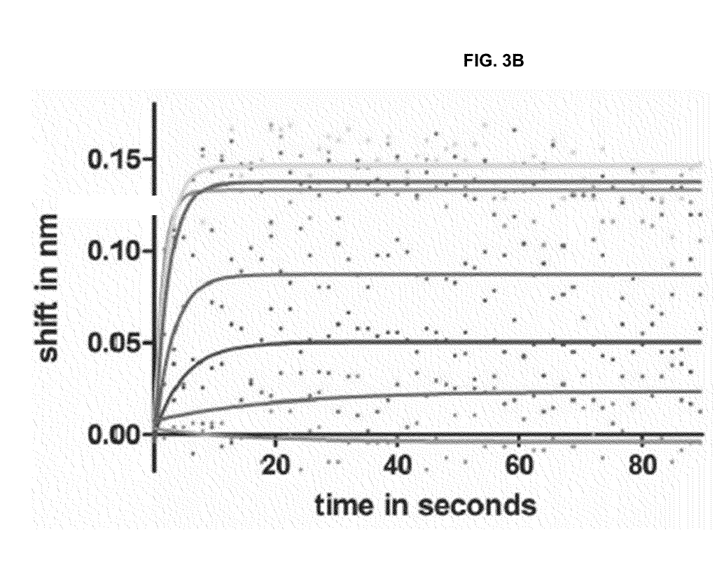

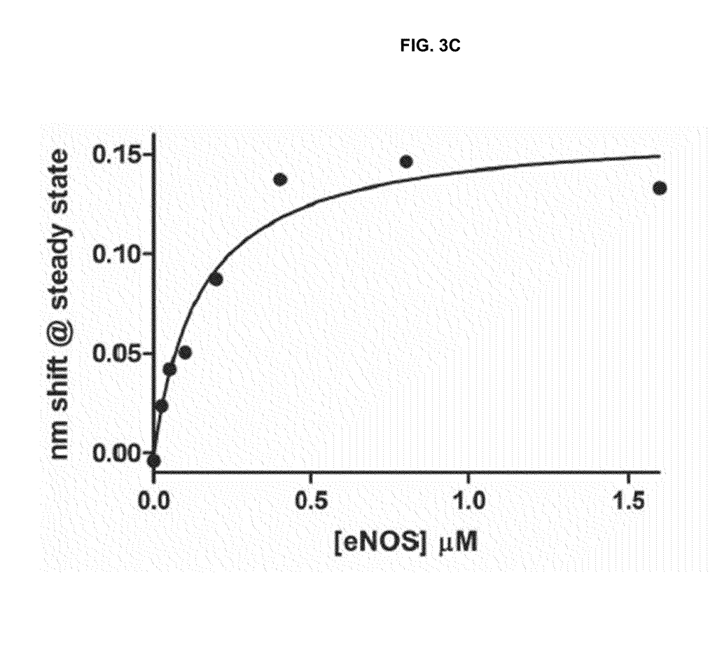

FIG. 2B shows sensorgrams of an eNOS concentration course. eNOS concentrations in nM units were 696 (open diamonds), 232 (squares), 77 (triangles), 26 (circles) and 0 (black diamonds). Fits to a two-component sequential model are shown as solid lines. Dissociation phases were similar to trace a (circles) in FIG. 1. Kinetics parameter sets for successive traces in order of decreasing eNOS were 0.1, 0.01, 0.006, 0.001; 0.03, 0.01, 0.0002, 0.001; 0.0008. 0.01, 0.006, 0.001; 0.0025, 0.01, 0.0002, 0.001 in sec-1 for k1, k2. k3, and k4. B. Log k1 (rapid phase) vs. log [eNOS] demonstrated first order binding. Points were determined from simulations shown in FIG. 3A. Error bars represented the range of k1 that produced acceptable fits. Solid line was first order binding. Dotted line was second order binding.

FIG. 2B trace b shows the binding of nNOS to p38. The low amplitude of the signal compared to eNOS indicated that binding was an order of magnitude weaker, and binding was 30 fold slower. In FIG. 2B trace a eNOS was present at saturating concentrations, while the same concentration of nNOS was below the Kd; similar binding of eNOS can be obtained at concentrations about thirty fold lower (see titration in FIG. 2). Weak association of nNOS to p38 may be through basic residues in the AI or the CaM binding region, or may represent a different mode of interaction.

Mutants of eNOS, where the control elements were deleted, were previously studied (28). Binding of a deletion mutant in which the autoinhibitory element has been removed to p38 is comparable to the binding of nNOS, but since the non-specific binding of the eNOS mutant is somewhat greater than that of wild type enzymes, it is not possible to draw more detailed conclusions about weak secondary binding sites. The results are consistent with the behavior of nNOS.

FIG. 2B trace c (triangles) shows p38 immersed in eNOS as in trace a except that excess Ca⁺²/CaM was present during association. CaM abolishes binding, suggesting that binding sites overlap, consistent with recognition of the pentabasic motif. p38-eNOS interaction can also be observed with eNOS as ligand and p38 as free analyte (data not shown). The signal is much smaller; this was expected because eNOS is a large protein (dimer molecular weight about 260 kDa) and p38 is much smaller (molecular weight about 41 kDa). Inhibition by CaM was also observed. Reversibility of ligand and analyte demonstrated that the interaction was not an artifact of immobilization.

FIG. 2B shows concentration dependence of eNOS binding to p38. Simulation with two components produces good fits; a sequential fit is shown, but good fits can also be obtained with two parallel components. The rapid association phase, most likely to be physiologically relevant, was insensitive to choice of model. The magnitude and rate of eNOS binding that most closely matched the binding of nNOS in FIG. 1 (b) was obtained at concentrations more than an order of magnitude lower.

Rapid binding is first order with respect to analyte (varied during titration), and the simulations are pseudo-first order with respect to immobilized ligand. Because the analyte was present in great excess, the observed binding kinetics are first order (in immobilized ligand) at each analyte concentration. At high eNOS concentrations the rate of binding (k₁) was much faster than the rate of release (k₂), hence the apparent rate constant k_{obs}˜k₁. The pseudo first order reaction rate at 232 nM eNOS was 0.03±0.003 sec⁻¹, corresponding to a diffusion limited rate constant of 0.13±0.01×10⁶ M⁻¹ sec⁻¹. K_{d} for eNOS binding to p38 was k₂/k₁, about 80±10 nM.

As shown in FIG. 2A, dissociation of eNOS from p38 was incomplete. This was not due to attainment of equilibrium in the rapid binding phase, because so little eNOS was bound to the sensor that full release would not produce a concentration in the dissociation buffer consistent with observable binding. These results were interpreted in terms of a sequential model in which the complex on the sensor was converted to a slowly released form. This process, occurring on the sensor over a time scale of several minutes, was unlikely to be biologically significant, but modeling it improved understanding of the fast phase.

eNOS bound to ERK. FIG. 3A shows the association phase of eNOS and nNOS binding to ERK-2. Immobilized ERK2 was immersed in 200 nM NOS at time 0. Trace a (circles) was eNOS binding. Trace b (squares) was nNOS binding. Solid lines indicate fits to a single exponential.

FIG. 3B shows sensorgrams of eNOS concentration course. eNOS concentrations in nM units were 1.6 μM (yellow), 0.8 μM (green), 0.4 μM (orange), 0.2 μM (purple), 0.1 μM (blue), 25 nM (red) and 0 (brown). Solid lines indicate fits to a single exponential.

FIG. 3C shows a plot of steady state amplitude against eNOS concentration. The fit shown is for K_{d}=160 nM.

FIG. 3A showed similar experiments demonstrating the binding of eNOS, but not nNOS, to ERK-2. The concentration dependence of eNOS binding to ERK-2 was shown in FIG. 3B. The concentration dependence was similar to that of the binding of eNOS to p38. The apparent K_{d} in the single component fit shown is about twice that measured for p38, but the data are consistent with K_{d} values as small as 90 nM (140±50 nM). A single kinetics component produces suitable fits for the initial 100 seconds of binding. Rapid binding is first order with respect to the analyte, and at each analyte concentration the observed kinetics are pseudo first order as in section 3.1. The pseudo first order reaction rate at 100 nM eNOS is 0.125±0.025 sec⁻¹, corresponding to a diffusion limited rate constant of 0.125±0.025×10⁶M⁻¹ sec⁻¹. Low amplitude, likely due to low binding activity of ligand, prevented accurate determination of off rates. Steady state analysis (FIG. 3C) of the binding in FIG. 3B yielded a K_{d} of 160 nM.

The effect of p38 binding on eNOS activity was investigated using the cytochrome c reduction assay for electron transfer and the hemoglobin capture assay for NO production. There was no significant effect on cytochrome c reduction. Weak inhibition of NO formation by 1 μM p38 in the presence of 0.6 μM CaM was observed; NO production was reduced by an average of 30%. This was consistent with competition between p38 and CaM, assuming that CaM is the stronger ligand as indicated in biosensor experiments. The protein-protein interaction alone does not appear to significantly affect eNOS activity.

The data demonstrated that p38 and ERK-2 bound eNOS with high affinity and bound nNOS only weakly or not at all. This identified the pentabasic sequence in the autoinhibitory element of eNOS, but not nNOS, as an important MAP kinase recognition site. Competition with CaM provided additional support for the pentabasic sequence as a MAP kinase target since these elements are proximally located in the three dimensional structure; displacement of the AI by CaM is the primary activating event in both eNOS and nNOS.

Physiological p38-eNOS binding has great potential in mediating signaling pathways known to involve both eNOS and p38. Other kinases might associate with the p38-eNOS complex rather than act as free intermediates. Trafficking of eNOS to cellular compartments is established (17-19), providing opportunity for differential interaction with p38 in some cell states. Evidence exists that MAP kinase pathways are involved in regulating both the expression and phosphorylation state of eNOS; some of these pathways inhibit and others activate NO synthesis (e.g., 30, 31) strongly suggesting a mixture of mechanisms that include pathway mediated effects and direct effects, e.g., direct phosphorylation of eNOS by ERK or p38, or activation of an intermediate activating kinase such as AKT in a multiprotein signaling complex.

Available evidence demonstrates that ERK directly phosphorylates eNOS in BAECs, and forms complexes with eNOS and additional components (11). These complexes are likely to be mediated at least in part by the interactions studied here. We point out that the location of the pentabasic motif, adjacent to the PKA phosphorylation site S633, suggests a mechanism of interaction between modes of inactivation and activation on the enzyme. Recently we directly confirmed inhibition of CaM binding after PKC phosphorylated T495, adjacent to the tribasic motif directly at the start of the CaM canonical target (21). It is of interest that the MAPK binding site is located in the AI adjacent to a comparable phosphorylation site; the MAPK site exposure is increased by AI displacement but MAPK competes with CaM, and the MAPK binding is positioned to interact with both PKA and AKT phosphorylation sites.

An alternative possibility with broader implications would involve additional partners. The pentabasic sequence of eNOS is recognized by a binding partner in mitochondria (32). Similar elements are involved in nuclear trafficking. It is easy to envision a system of alternating partners bearing base-rich sequences and their recognition sites that would enable kinases activated by MAP kinases to interact with partners and in turn, eNOS. Such interactions could create a phosphorylation cascade, but could also be the basis for inhibition. We are now investigating the potential of eNOS-MAPK interactions for enhancement of eNOS phosphorylation and inhibition of MAPKAP-2/3 kinases.

ERK directly phosphorylates eNOS in BAECs, and forms complexes with eNOS and additional components (11). These complexes are likely to be mediated at least in part by the interactions reported herein. The location of the pentabasic motif, adjacent to the PKA phosphorylation site S633, suggested a mechanism of interaction between modes of inactivation and activation on the enzyme. The inventors recently confirmed inhibition of CaM binding after PKC phosphorylated T495, adjacent to the tribasic motif directly at the start of the CaM canonical target (21). The MAPK binding site is located in the AI adjacent to a comparable phosphorylation site; the MAPK site exposure is increased by AI displacement but MAPK competes with CaM, and the MAPK binding is positioned to interact with both PKA and AKT phosphorylation sites.

The endothelial nitric oxide synthase bound MAP kinases at a pentabasic site in the unconserved region of the autoinhibitory insertion associated with the eNOS FMN binding domain. No potential MAP kinase phosphorylation sites are associated with this region in eNOS, but many good candidates elsewhere in the eNOS structure may be accessible to a kinase bound at the pentabasic site. MAP kinases bind eNOS, but not homologs lacking the pentabasic site, with about 100 nM affinity and a forward rate constant of about 1.3×10⁵ M⁻¹ sec⁻¹. Calmodulin forms a ternary complex that weakly promotes dissociation of p38 from eNOS. The eNOS-MAP kinase interaction may provide a scaffold for the formation of larger complexes with additional components, including Akt and Raf-1.

eNOS was inhibited by MAP kinase. eNOS expression and purification were performed as previously described. Prosthetic group content was measured spectrophotometrically. NO synthase activity was measured using hemoglobin capture, and reductase activity was measured using cytochrome c reduction. ERK2 was purchased from SignalChem (Richmond, British Columbia Canada).

ERK kinase reactions were performed in 20 mM Hepes (pH 7.4), 1 mM DTT, 10 mM MgCl₂, 1 mM ATP, 10% glycerol, and when using the high concentration eNOS, 0.75 mM EGTA. eNOS was used at various concentrations (45 μM to 1.6 μM for fluorescence and eNOS/cytochrome C experiments, respectively). Reactions were done plus or minus ERK (0.12 μM to 0.01 μM, depending on eNOS concentration) at room temperature as described for the figures, and were ‘stopped’ by putting on ice until activity was tested, within 5 hours. 0.315 nMoles of eNOS phosphorylated and un-phosphorylated were analyzed by mass spectroscopy of trypsin digested enzyme (Emory University proteomics facility) with duplicate phosphorylated and unphosphorylated samples.

Fluorescence lifetime measurement was obtained with time-resolved intensity decays recorded using a PicoQuant Fluotime 100 time-correlated single-photon counting (TCSPC) fluorescence lifetime spectrometer as described (42). FMN was excited at 473 nm using a pulsed laser diode with 20 MHz repetition rate; experiments with 378 nm excitation produced similar results. The decay of fluorescence can be represented as the sum of individual exponential decays:

$$I ( t ) = {\sum }_{ii = 1}^{n} exp ( - t / {τ}_{i} ) \qquad ( 1 )$$

where the τᵢ are the decay times and αᵢ are the amplitudes of the i^{th} component. The fractional contribution of the i^{th} component in the steady-state is:

$${f}_{i} = \frac{{α}_{i} {τ}_{i}}{{\sum }_{j} {α}_{j} {τ}_{i}} \qquad ( 2 )$$

Individual values of πᵢ and τᵢ were determined from simulation with PTI's Felix GX software with PowerFit 10 simulation module, using deconvolution of an instrument response function obtained from scattering and nonlinear least squares fitting to multiple exponentials. The quality of the fits were characterized by χ² (48).

For optical biosensing, biolayer interferometry (BLI) experiments were conducted essentially as described. Briefly, biotinylated CaM was immobilized on streptavidin sensors. After establishing a baseline in binding buffer (10 mM HEPES, pH 7.4, 100 mM NaCl, 10% glycerol, 10 μM CaCl₂, 0.05% Surfactant P-20), sensors were moved to binding buffer containing eNOS for 180 s. Sensors were then moved to buffer only to monitor dissociation for 180 s. Nonspecific binding was measured by immersing sensors without CaM in analyte and was negligible in all cases.

Surface plasmon resonance (SPR) experiments were conducted on a Biacore X100 instrument using a biotin CAPture chip. Biotinylated CaM, about 150 RU in all cases, was immobilized prior to single-cycle kinetics performed in binding buffer.

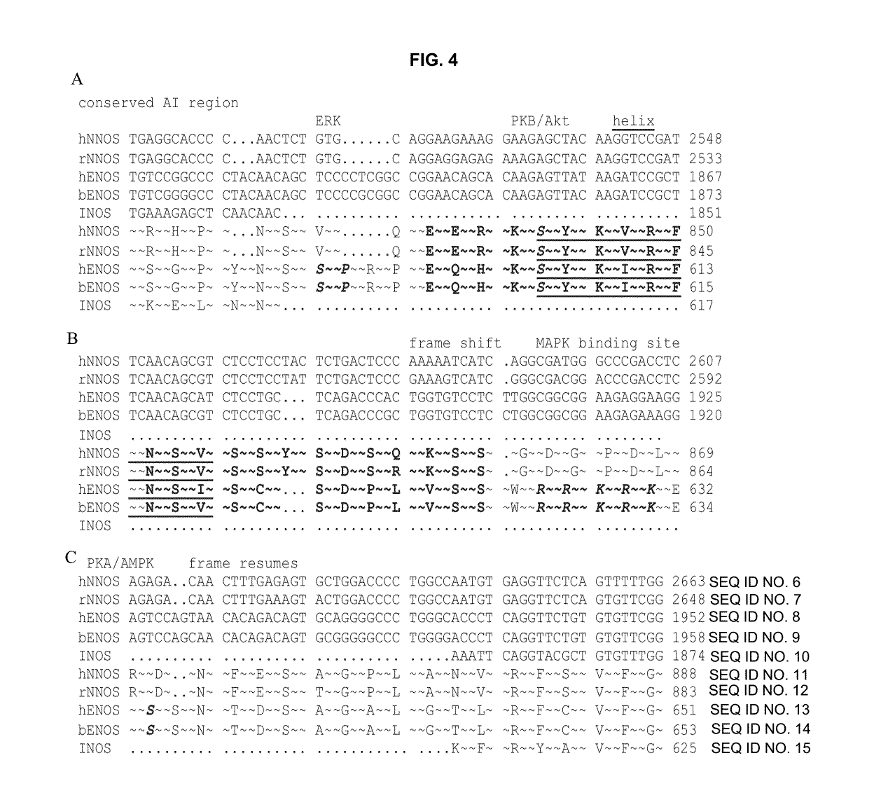

FIG. 4 shows the alignment of the DNA and amino acid sequences of mammalian nitric oxide synthase isoforms in the autoinhibitory element region, a region corresponding to an ab turn within the Rossman fold FMN binding domain. The conserved region (bold) and frame shifts caused divergence of the variable region in eNOS and nNOS. Helix residues are underlined. Phosphorylation sites for ERK, Akt, and PKA/AMPK, and the binding site for MAP kinases (ERK, P38) are marked with bold italics. Human sequences are denoted by h, rat sequences by n, and bovine sequences by b. All DNA sequences are numbered from the first base of the start codon in the most commonly studied splice form. DNA accession numbers: hnNOS, D16408; rnNOS, X59949; heNOS, M93718; beNOS, M99057; hiNOS, L24553. Protein accession numbers: hnNOS, P29475; rnNOS, P29476; heNOS, P29474; beNOS, P29473; hiNOS, P35228.

The signal generators eNOS and nNOS differ from the cytokine induced isoform iNOS in that they have an extended insertion at this point that serves as an autoinhibitory element (AI) displaced by calmodulin binding to a spatially adjacent site.

The N terminal half of the AI, shown in bold, contains a conserved helical region (underlined residues) that locks down the FMN binding domain through hydrogen bonds to both the FMN binding domain and the NADPH binding domain. This prevents conformational changes that are an obligatory part of the catalytic cycle. S617 and S635 in bovine eNOS (S615 and S633 in human eNOS) are phosphorylated by kinases (AKT and PKA) that activate the enzyme.

As described above, MAP kinases bind to a pentabasic sequence present in eNOS but not nNOS. This site, shown in bold italic, confers 50-100 picomolar affinity for p38 and ERK. The location in this regulatory region suggests interactions between MAP kinases and other modulators of eNOS activity.

The evolutionary origin of the MAP kinase binding site is implied by the frame shift in the eNOS sequence in comparison to nNOS at the end of the conserved AI region. A compensating shift restores the reading frame 27 bases later in eNOS, and homology at the amino acid level immediately resumes. During the 27 base pair frame shift, however, there is no relationship between eNOS and nNOS at the amino acid level; some homology appears to persist at the DNA level. Divergence of eNOS from nNOS occurred during or before the development of amphibians, and available amphibian sequences have either pentabasic or tetrabasic motifs. It is plausible that these motifs arose by chance and were stabilized during evolution by protein-protein interactions.

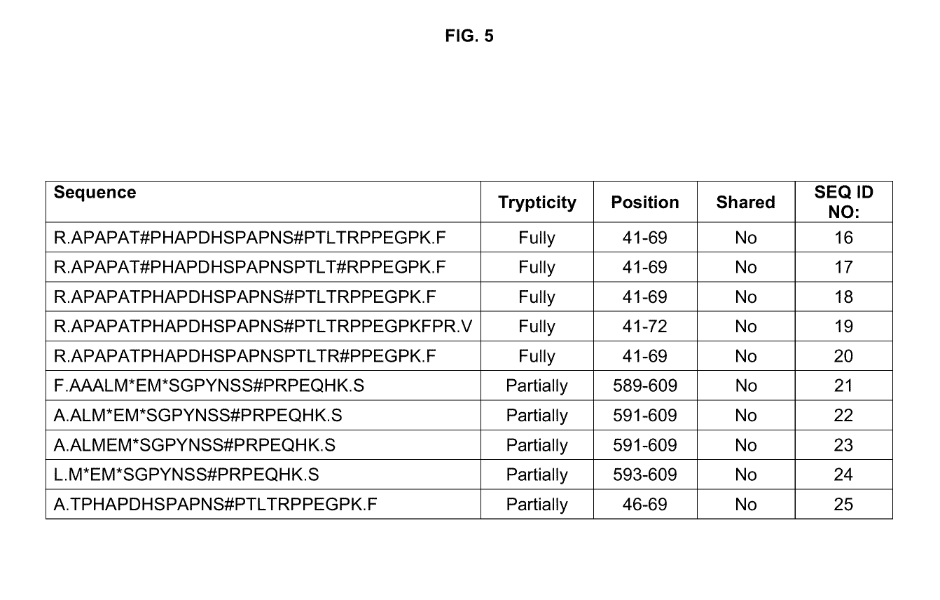

Phosphorylation of eNOS by ERK was detected in vitro by measurement of ADP. The results showed that over a one hour incubation two or three ADP were formed per eNOS monomer, suggesting that multiple sites were phosphorylated. FIG. 5 shows the results of mass spectroscopy of eNOS tryptic digests of unphosphorylated eNOS and eNOS phosphorylated for 15 minutes by ERK, showing phosphorylated peptides consistent with the known specificity of ERK. Phosphorylation sites are indicated by a hatch (#), and digestion locations are indicated by periods (.). Because ERK phosphorylates TP and SP motifs, the results are uniquely consistent with S602, T46, and S58 phosphorylation. Asterisks indicate oxidized methionines. No peptides were shared with the unphosphorylated control.

Coverage was essentially complete; 35 phosphorylated tryptic peptides were obtained in addition to 3545 unphosphorylated peptides.

S602 and T46 were identified unambiguously as phosphorylation sites from the mass spectroscopy results alone. The data showed that either S58 and T46 was phosphorylated; because MAP kinases phosphorylate at SP or TP sites, the third position was unambiguously identified as T62. No indication of phosphorylation on other sites was seen.

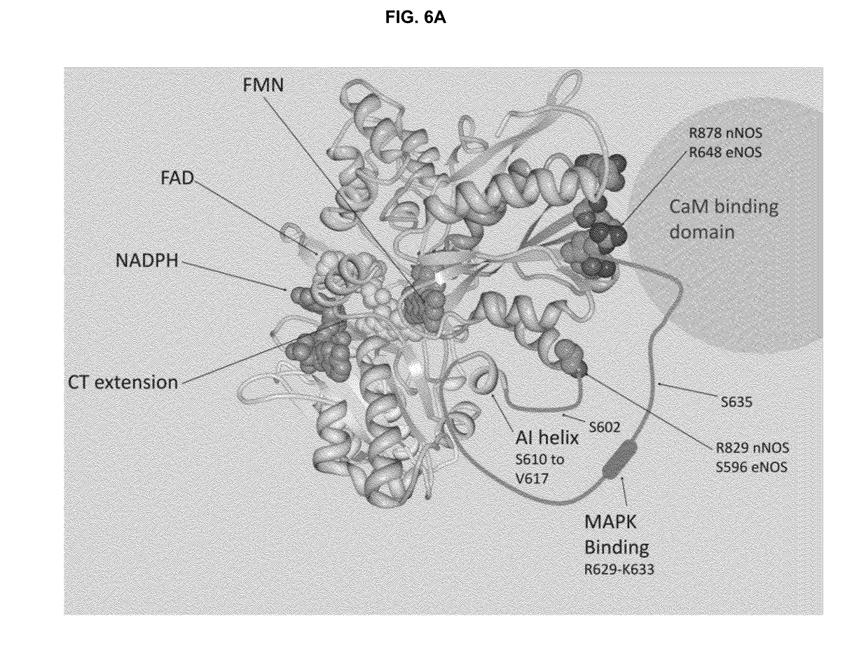

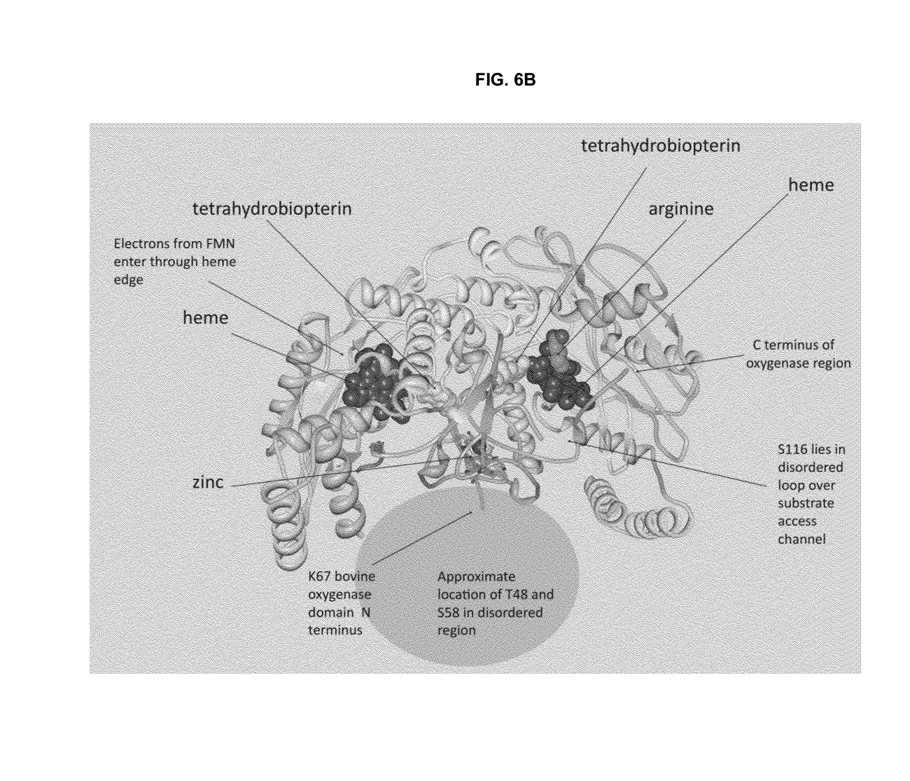

FIG. 6A is a structural cartoon of NOS reductase domains based on the crystal structure of nNOS reductase construct. The FMN binding domain is blue, the FAD binding domain green, and the NADPH binding domain is tan. Cofactors are shown in solid render, and the residues at the N terminal edge of the FMN binding domain and at the ends of the autoinhibitory insertion (R878/R648 and R829/S596 for nNOS/eNOS) are marked by solid render. The approximate extent and position of the disordered regions of the AI are indicated by a drawn coil. The autoinhibitory element of nitric oxide synthase (AI) carried previously identified targets for Akt and PKA phosphorylation as well as a MAP kinase binding site and a target for ERK phosphorylation (S602) that also matched the target motifs of other MAP kinases. The C terminal extension carries S1179, a target for Akt and PKA (see PDB 1TLL).

FIG. 6B is a structural cartoon of eNOS oxygenase dimer based on crystal structures. The direction of the backbone is indicated by color, with the N terminal blue and the ribbon shading through green to tan at the C terminal. Cofactors and substrate arginine are shown in solid render, with heme shown in red and tetrahydrobiopterin in yellow. The location of two ERK targets in a disordered region at the bottom of the figure is indicated; the region is near the dimer interface. The C terminal end of the domain is shown emerging from the face at right; this becomes the calmodulin binding site, and after a short connector joins the FMN binding domain shown in FIG. 3A. This domain must supply electrons to the heme at right by disengaging from the reductase complex and re-orienting (see PDB 4NSE and 3NOS). FIG. 6A shows the structure of the reductase portion of nNOS, corresponding roughly to the C terminal half of the enzyme. No corresponding eNOS crystal structure is available, but the two enzymes are highly homologous in this region and the conserved structures shown should be nearly identical.

The ribbon diagram traces the path of the backbone through the FMN, FAD, and NADPH binding domains, with the cofactors shown in solid render. In this conformation the FAD and FMN isoalloxazines are in Van der Waals contact. At the opposite edge of the FMN binding domain beta sheet, the ends of the AI are exposed at the adjacent ends of an alpha helix and a beta strand; the trace appears discontinuous because the chain is too flexible here to have a well-defined structure. The AI helix is visible as a disconnected feature, and is connected to the FMN domain beta sheet by a long disordered loop on the C terminal side and a short disordered loop on the N terminal side. The long loop carries the MAP kinase binding site close to the edge of the beta sheet and the adjacent CaM binding site, accounting for the CaM-MAP kinase competition we previously reported. S602 is located in the short disordered loop directly adjacent to the alpha helical region that precedes the AI. S617 is located in the AI helix, and S635 is in the long disordered loop between the MAP kinase binding site and the beta strand that follows the AI.

FIG. 6B shows the structure of the eNOS oxygenase dimer. The heme and tetrahydrobiopterin cofactors are shown in solid render along with the zinc atom that stabilizes the dimer. T46 and S58 are located in a disordered region adjacent to the loops bearing the cysteine residues that coordinate the zinc; the sites of myristoylation and palmitoylation are further towards the N terminal. This region of the oxygenase domain surface is close to the point where the polypeptide chain leaves the oxygenase domain to form the CaM binding site, and also reasonably close the site of heme reduction on the opposite monomer.

Table 1 summarizes experimental results showing the effect of ERK mediated phosphorylation on eNOS activity in NO synthesis and cytochrome c reduction. NO synthesis measured spectrophoto-metrically through the reaction of NO with oxyhemoglobin was inhibited by 50%. Reduction of ferricytochrome c by eNOS was monitored at 550 nm to assess the effect of phosphorylation on electron transfer within the reductase unit. Phosphorylation inhibited cytochrome c reduction by about 50%.

TABLE 1

| | NO synthesis | Cytochrome | Fluorescence lifetime | | |
|---|---|---|---|---|---|
| | nm/min/mg | c reduction | state populations | | |
| | enzyme | min¹ | 90 ps | 0.9 ns | 4.3 ns |
| Unphosphory- | 110 (10) | 143 (20 | 53.45 | 20.21 | 26.35 |
| lated eNOS | | | | | |
| ERK phosphor- | 42 (7.6) | 55 (5) | 85 | — | 15 |
| ylated eNOS | | | | | |

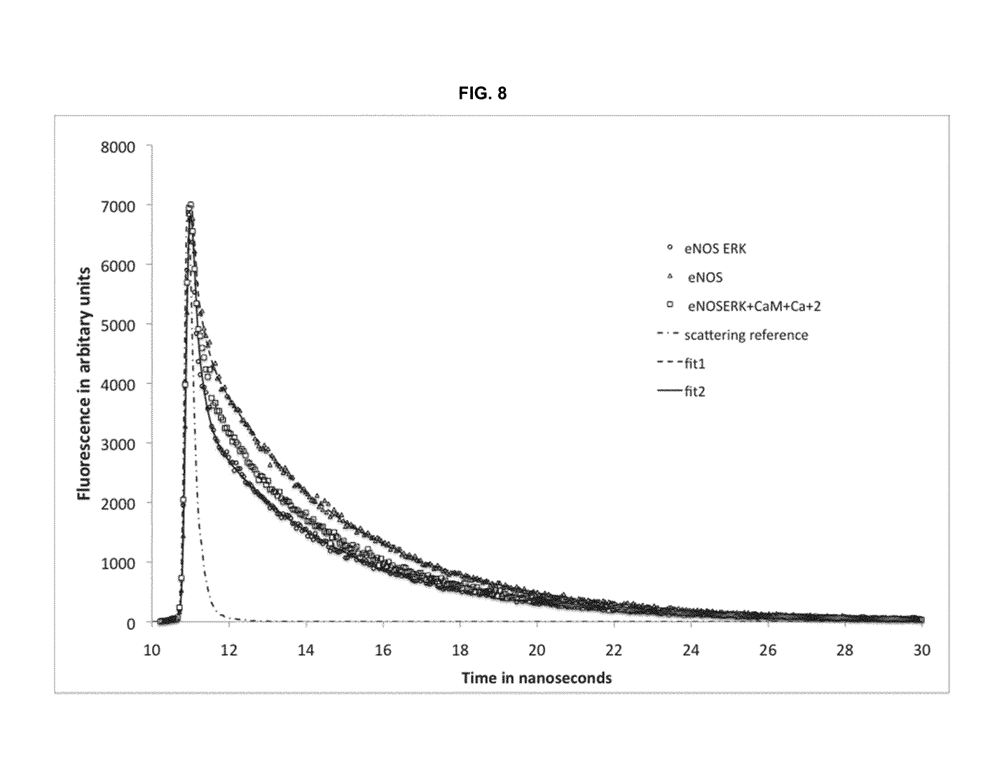

Effects of ERK catalyzed phosphorylation of eNOS on activity and on the population of eNOS conformational states characterized by FMN fluorescence lifetimes. The lifetime distributions in Table 1 differ somewhat from those in the fits shown in FIG. 8 because they are derived from decays of calmodulin activated enzyme to correspond to NO synthase activity. The distributions in FIG. 8 show the effects of phosphorylation in the absence of calmodulin.

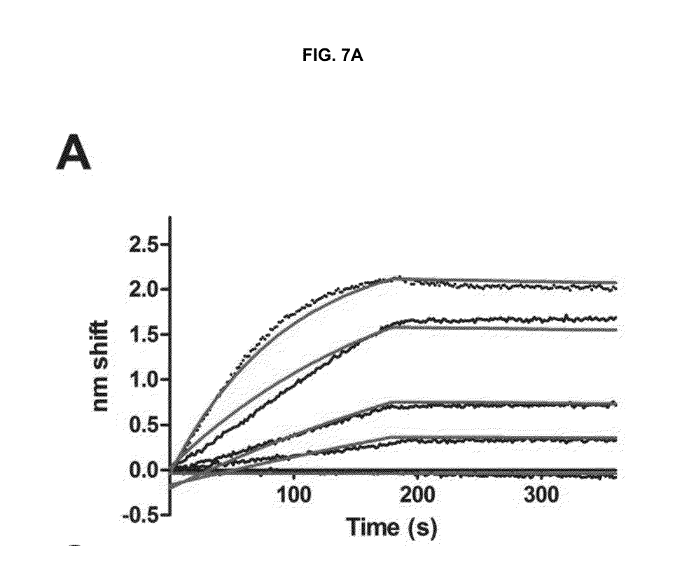

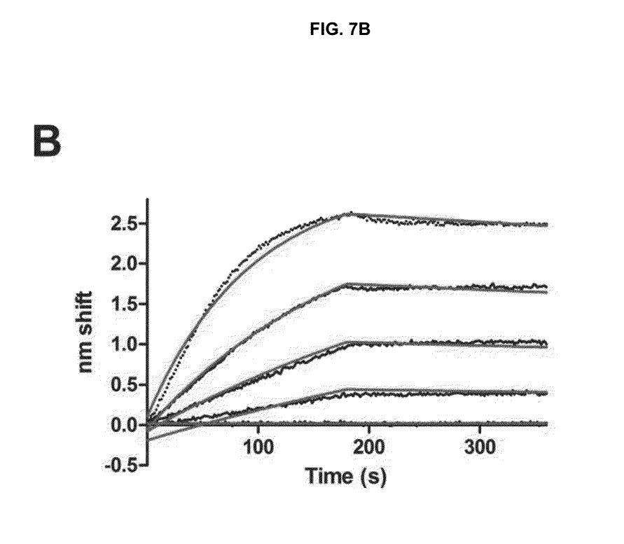

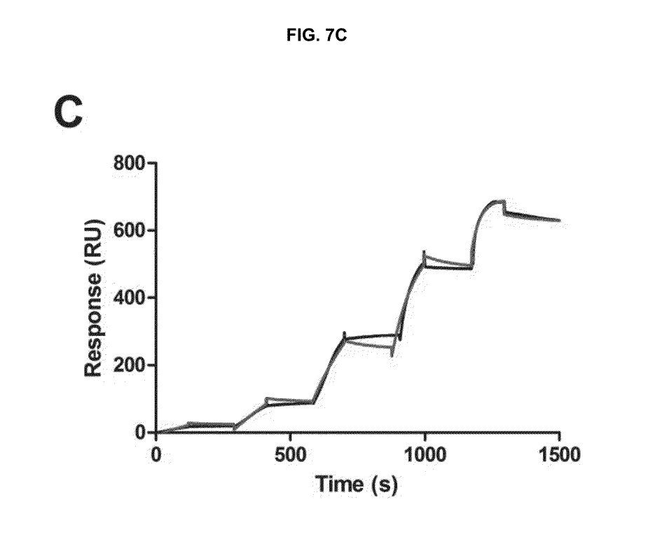

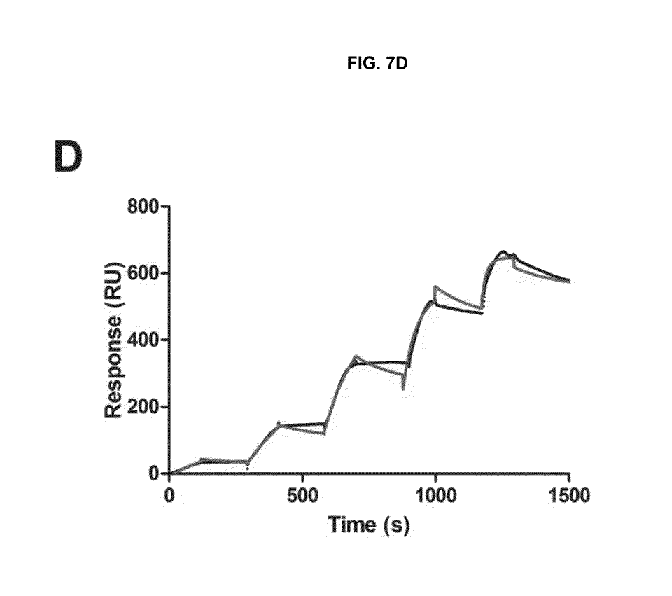

The inventors previously showed that CaM binding to unphosphorylated eNOS was diffusion limited and that PKC inhibition of eNOS via phosphorylation at T497 strongly inhibited CaM binding. ERK phosphorylation of the target residues does not strongly affect CaM binding, suggesting that PKC and ERK inhibit eNOS by different mechanisms. Optical biosensing experiments demonstrated near −1 nM affinities regardless of phosphorylation state. FIGS. 7A and 7B show BLI traces with fits to global single-state models for CaM binding to unphosphorylated and phosphorylated eNOS, respectively. Rate constants are shown in Table 2.

TABLE 2

| | Unphosphorylated | Phosphorylated |
|---|---|---|
| BLI (single state) | | |
| KₒₙM¹ S¹ | 1.2 × 10⁵ | 1.2 × 10⁵ |
| K_{off s}⁻¹ | 1.1 × 10⁻⁴ | 1.2 × 10⁻⁴ |
| K_{D}, pM | 920 | 2700 |
| SPR (Sequential model) | | |
| Kₒₙ₁M¹ S¹ | 6.4 ×. 10⁴ | 1.1 × 10⁵ |
| K_{off 1} S⁻¹ | 2.0 × 10⁻³ | 2.5 × 10⁻³ |
| K_{on 2} S⁻¹ | 8.4 × 10⁻³ | 3.7 × 10⁻³ |
| K_{off 2} S₁ | 1.9 × 10⁻⁴ | 1.1 × 10⁻⁴ |
| K_{D}, pM | 720 | 650 |

At the concentrations examined binding approximates single state with remarkably similar profiles regardless of phosphorylation. The difference in affinity observed was entirely due to a three-fold higher k_{off}, although this may not be physiologically relevant.

FIG. 7 shows an optical biosensing analysis of CaM-eNOS binding. FIG. 7A is a BLI sensorgram of CaM binding to 0 nM, 10.9 nM, 31.9 nM, 43.7 nM, and 87.5 nM unphosphorylated eNOS. Raw data are in black. Fits to a single-state association-then-dissociation model are shown in red. FIG. 7B show the same as in FIG. 7A, but for the same concentrations of phosphorylated eNOS. FIG. 7C is a SPR sensorgram of single-cycle kinetics of tethered CaM binding to 6.2 nM, 18.5 nM, 55.6 nM, 167 nM, and 500 nM analyte unphosphorylated eNOS. Reference-subtracted raw data are in shown in black along with fits to a two-state sequential model in red. FIG. 7D show the same as in FIG. 7C, but with phosphorylated eNOS.

Single-cycle kinetic analysis by SPR also revealed that CaM binding of unphosphorylated eNOS (FIG. 7C) was highly similar to phosphorylated (FIG. 7D). CaM was immobilized prior to injection of analyte eNOS. Fits to a sequential model (A+B<->AB<->AB*) generated K_{D}s similar to those determined from BLI (Table 1), indicating that the secondary event was a minor component.

The inventors recently showed that iNOS passes through a series of obligatory conformational states during its catalytic cycle, including an input state in which the FMN binding domain is closely associated with the FAD and NADPH binding domains, an output state in which the FMN binding domain is associated with the heme containing oxygenase domain, and a series of open conformations in which FMN is not closely coupled to other prosthetic groups. These states can be resolved by their very different fluorescence lifetimes; eNOS and nNOS have similar conformational states. Calmodulin activation of eNOS and nNOS resulted in increased levels of the output and open states relative to the closed input state.

FIG. 8 shows fluorescence decays of unphosphorylated and ERK phosphorylated eNOS holoenzyme FMN excited at 473 nm and detected at 525 nm in the presence and absence of calmodulin. The eNOS concentrations in the samples were 1 μM, and were from the same preparation. ERK phosphorylation favors conformational states with shorter lifetimes; calmodulin activation produces the opposite effect (not shown). Calmodulin partially reverses the effect of ERK phosphorylation, but the effect of calmodulin binding to ERK phosphorylated NOS was much smaller than the effect of calmodulin on the unphosphorylated enzyme. Fitting parameters were as follows. For unphosphorylated eNOS 90 ps, 78%; 3.65 ns, 20%; 0.9 ns, 0.68%, 10.1 ns, 1.6%; c²=1.10. For ERK phosphorylated eNOS: 80 ps, 86%, 3.83 ns, 12.4%, 1.1 ns 1.8%. All experiments were performed six times using at least two different preparations. The scattering reference was collected at 475 nm, and indicated the width of the exciting pulse.

The invention also discloses antibodies to detect eNOS phosphorylation. In embodiments, the antibodies detect eNOS phosphorylation by ERK or other kinases at positions that include, but are not limited to, S602 in bovine eNOS, S600 in human eNOS, T46 in bovine eNOS, T44 in human eNOS, S58 in bovine eNOS, S56 in human eNOS, and S116 in bovine eNOS, S114 in human eNOS. In one embodiment, these antibodies may be generated against phosphorylated, e.g., by ERK, synthetic peptides with sequences derived from the region around the site.

The term antibody includes a polyclonal antibody, a monoclonal antibody, a chimeric antibody, a single chain antibody, a humanized antibody, a Fab fragment, a F(ab′)₂ fragment, and fragments produced by a Fab expression library, as known in the art. Antibodies can be fragmented using conventional techniques and the fragments screened for utility in the same manner as described for whole antibodies. For example, F(ab)₂ fragments can be generated by treating the antibody with pepsin. The resulting F(ab)₂ fragment can be treated to reduce disulfide bridges to produce Fab fragments. The disclosed antibody also includes bispecific and chimeric molecules having affinity for eNOS conferred by at least one complementarity-determining regions (CDR) of the antibody.

Antibodies that specifically bind eNOS and/or phosphorylated eNOS can be used in a variety of methods known in the art. These include, but are not limited to, immunohistochemical staining of tissue samples to evaluate the abundance and pattern of eNOS and/or phosphorylated eNOS. Anti-phosphorylated eNOS antibodies can also be used to measure the effect of kinases and/or signal transduction pathways that affect kinase activity on eNOS phosphorylation.

In embodiments, the antibody is labeled with a detectable marker. The detectable marker may be, e.g., a chemiluminescent moiety, an enzymatic moiety, a fluorescent moiety, a radioactive moiety, combinations of these, etc.

In one embodiment, the antibody is directed to a region comprising S602 of eNOS. The S602 antibody specifically recognizes phosphorylation of the serine residue at position 602 (pS602). Specifically, the antibody was prepared as follows: the phospho-peptide was linked to keyhole limpet hemocyanin and was used to immunize rabbits. The resulting antibodies were affinity purified by NeoBioLab (Woburn, Mass.). (See “Endothelial nitric oxide synthase is regulated by ERK phosphorylation at Ser602”, Salerno et al., Biosci Rep. 2014; 34(5)).

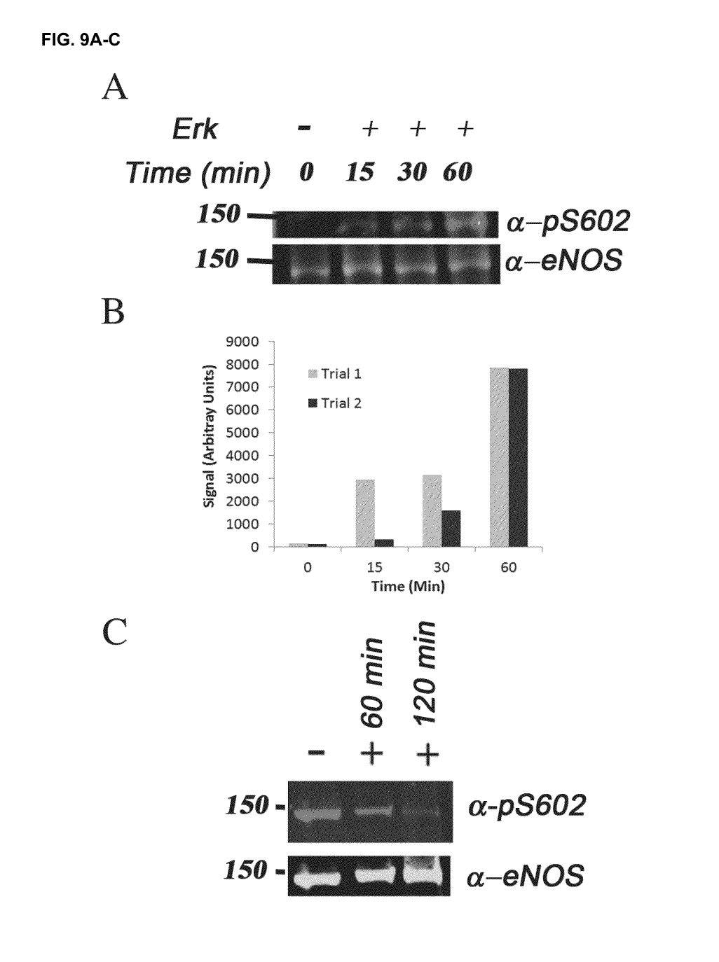

As shown in FIG. 9A, in the absence of Erk kinase (“-”), the anti-pS602 antibody (α-pS602; top panel) shows minimal reactivity, while a generic eNOS antibody (α-eNOS; lower panel) shows the presence of the purified eNOS. Westerns were performed as described in Salerno et al., Biosci Rep. 2014; 34(5), with anti-eNOS antibody from Invitrogen (Clone 9D10), anti-pS602 antibody as described herein, and anti-pS116 antibody from AbCam, where Western blot analysis was conducted with Li-Cor secondary antibodies and results were visualized with the Li-Cor Odyssey. When the purified eNOS was incubated with Erk kinase for the indicated time period (0 min, 15 min, 30 min, 60 min), the anti-pS602 antibody specifically recognized Erk phosphorylation of purified eNOS. FIG. 9B summarizes results from two such experiments. To further confirm the specificity of anti-pS602 antibody, lysate from bovine endothelial cells which contained phosphorylated eNOS was treated with or without phosphorylase B, which enzymatically removes phosphate, and then probed with both the anti-pS602 and anti-eNOS antibodies. The results are shown in FIG. 9C: reactivity of the anti-pS602 antibody decreased in a time-dependent manner with phosphorylase B incubation, while reactivity of the generic anti-eNOS antibody remained constant. The data indicated and confirmed phospho-specificity of the inventive anti-pS602 antibody for eNOS that was phosphorylated at S602.

One embodiment is a method to determine if a biological sample contains a phosphorylated form of NOS. In this embodiment, the biological sample is contacted with an antibody that selectively binds to a phosphorylated form of NOS, but does not bind to a non-phosphorylated form of NOS. Binding is then assessed. Binding of the antibody to the biological sample indicates that the biological sample contains the phosphorylated form of NOS.

One embodiment is a method for screening a compound or agent for its efficacy to phosphorylate NOS. The compound or agent is combined with non-phosphorylated NOS to form a mixture under conditions suitable for the compound to phosphorylate NOS. The mixture is then contacted with an antibody that selectively binds to a phosphorylated form of NOS but not to a non-phosphorylated form of NOS. Phosphorylation of NOS is then assayed in the mixture. NOS phosphorylation indicates that the compound phosphorylates NOS.

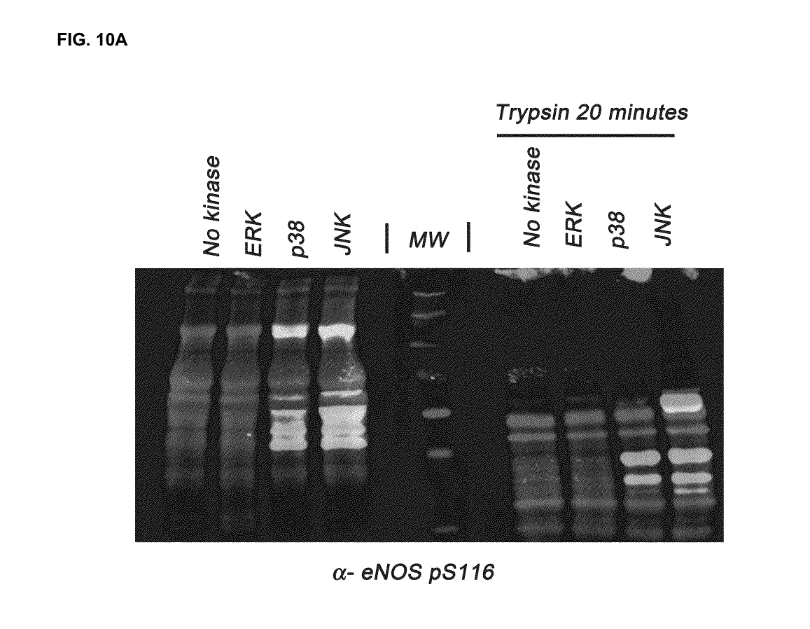

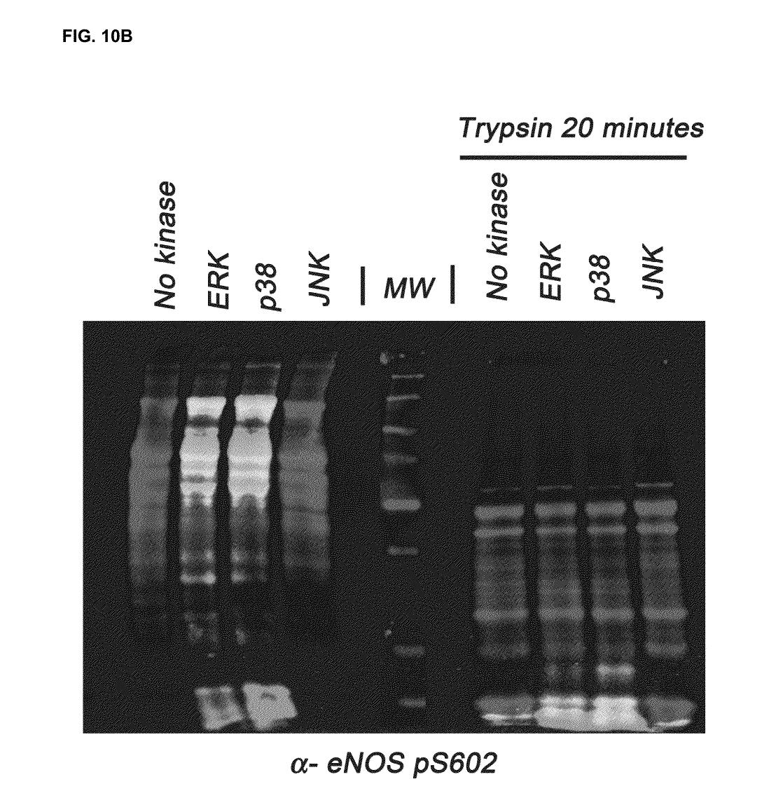

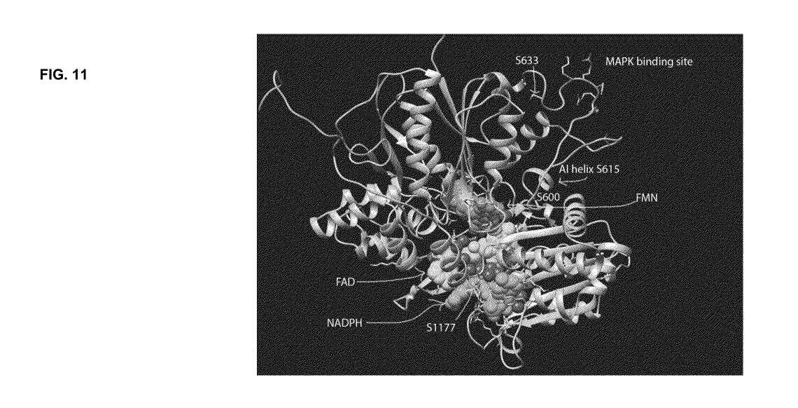

The inventors examined the ability of the anti-pS602 antibody and the anti-pS116 antibody to detect phosphorylation of eNOS by several different kinases. Western blots were performed as previously described. As shown in FIGS. 10A and B, purified eNOS was reacted with the kinases Erk, p38, and Jnk. The anti-pS116 antibody (a-eNOS pS116; FIG. 10A; green) and anti-pS602 antibody (a-eNOS pS602; FIG. 10B; green) was used to probe either full length purified eNOS (left side of panels) or trypsinized fragments of eNOS (right side of panels); the gels were also probed with a generic anti-eNOS antibody (red). In both FIGS. 10A and B, left side, the top yellow band indicates binding of both the phospho-specific and generic antibodies to the full length purified eNOS, where the combination of red plus green resulted in yellow. Both the anti-p602 and the anti-pS116 antibodies showed phosphorylation of full length enzyme, but also show that Jnk phosphorylation was not detected by anti-pS602, and Erk phosphorylation was not detected by anti-pS116. The results demonstrated that the kinases Jnk and p38 phosphorylated S116, and the kinases Erk and p38 phosphorylated S602. These results are consistent with mass spectrometry results for Erk. This further substantiated the specificity of the anti-pS602 antibody.

In FIGS. 10A and B, right side, the trypsinized samples, created by treating half the kinase reaction as described in reference 16, show completely different patterns on the gel. The eNOS oxygenase domain is relatively trypsin resistant, and shows up well with the anti-pS116 antibody at the 50 kD in the JnK sample, along with two major cleavage fragments under 37 kDa (FIG. 10A). The anti-pS602 antibody detected a peptide of about 12 kD-15 kDa (FIG. 10B), which is the region between the CaM binding domain and the auto-inhibitory element of eNOS, the two most easily cleaved sites on the enzyme.

ERK phosphorylation caused an increase in the input state, which has a lifetime of 90 ps because of close coupling between FMN and FAD, and a concomitant increase in the output state (0.9 ns lifetime) and the open states (4.3 ns average lifetime). This effect is in opposition to the effect of calmodulin binding, and accounts for the inhibition of NO synthesis and cytochrome c reduction by ERK mediated phosphorylation.

The effects of ERK phosphorylation on the activity of eNOS, and on the distribution of conformational states characterized by their FMN fluorescence lifetimes, is summarized in Table 1. The effects of ERK phosphorylation on the conformational manifold are opposite those of the principal activator, calmodulin. Inhibition of both NO synthesis and cytochrome c reduction was at least 60%, and may be much greater because eNOS could not be completely phosphorylated without long incubations that damage the enzyme.

The MAP kinases ERK and P38 are believed to participate in negative feedback signaling networks with eNOS, and good evidence has been obtained for direct phosphorylation of eNOS by ERK in BAECs. Based on indirect evidence obtained in intact cells, several sites have been proposed for ERK phosphorylation including S116, T497, and S635. Of these sites, only S116 has the SP/TP motif generally associated with MAP kinase targets. S116 is located on the oxygenase domain, and the MAP kinase binding site is located on the autoinhibitory element associated with the FMN binding domain.

Direct in vitro phosphorylation of purified eNOS with purified ERK confirmed that eNOS is a substrate for ERK, and further showed that phosphorylation did not require additional scaffolds or adapters. ERK phosphorylation inhibits both NO synthesis and cytochrome c reduction. This indicated that phosphorylation interfered with electron transfer reactions mediated by FMN.

Three sites of phosphorylation on eNOS were identified. S602, at the N terminal end of the AI, is spatially adjacent to both the MAP kinase binding site and the CaM binding site, and is well positioned to interact with other control sites. A ‘lockdown’ of the FMN binding domain was suggested by fluorescence results showing that phosphorylation pulls the enzyme's conformational distribution toward the input state. This accounts for the observed inhibition, and suggests that the negatively charged phosphate group interacts with positively charged residues to stabilize the input conformation. CaM appears to bind strongly to ERK phosphorylated eNOS, but was unable to effectively override phosphorylation imposed inhibition. Although the results of CaM activation and ERK phosphorylation on the fluorescence profile are fortuitously opposite, effects on the conformational equilibria are secondary to the changes in rates for conformational transitions. It is possible to inhibit the enzyme by locking it into any conformation, because the mechanism depends on conformational cycling.

The complex kinetics of CaM-eNOS binding are disclosed. ERK-phosphorylated eNOS demonstrated a highly similar CaM-binding profile: diffusion-limited association, very slow dissociation, and picomolar affinity. A threefold slower dissociation rate constant in BLI accounted for all of the affinity difference between unphosphorylated and phosphorylated eNOS. The difference may be kinetically significant (95% confidence intervals for k_{off} do not overlap) but cannot account for differences in regulation of NOS activities. Other factors, e.g. aging of complexes on the BLI sensors, may vary with phosphorylation and account for the observed differences. Indeed, the difference in k_{off} was much less pronounced in SPR and the affinities were nearly identical to the 650 μM.

ERK and p38 phosphorylation of eNOS had no measurable effect on the binding of eNOS to calmodulin. In contrast, phosphorylation of T497 by PKC interfered with CaM binding. The modes of ERK and PKC inhibition are thus entirely different: PKC prevented the binding of an activator, while ERK interrupted the catalytic cycle.

ERK also phosphorylates eNOS at T46 and S58; these residues are located in a disordered region on the surface of the oxygenase domain. Because ERK phosphorylates all three residues, their effects cannot yet be separated. The location of S602 is ideal for the control of FMN mediated electron transfer, and it lies close to the D domain type MAP kinase binding site. The mode of action of T46 and S58 phosphorylation is unclear; because the disordered N terminal region is known to participate in protein-protein interactions and protein trafficking, these sites may be signals that affect protein complex formation, myristolation/palmitoylation, and targeting to specific cellular compartments rather than direct regulators of activity, but they could also affect oxygenase domain function. It is unclear that ERK bound to the AI pentabasic site can phosphorylate sites on the oxygenase domain. Given that both the AI loop that carries this site and the target region are flexible, this may not be impossible, but it is possible that a secondary biding site exists that facilitates phosphorylation at T46 and S58.

It is unlikely that MAP kinases phosphorylate residues such as T497, S617, S635, and S1179. All these sites, with the exception of T497, activate eNOS, and none of them has an SP or TP target motif. When phosphorylation of these sites correlates with MAP kinase activation, it is likely that this occurs in a pathway dependent manner. For inhibitory events such as T497 phosphorylation, this might represent parallel pathways of inhibition. For activating events, it probably represents feedback pathways involved in push-pull regulation.

S116 was not detected after ERK phosphorylation in vitro. This residue is located on a flexible loop near the mouth of the substrate access channel on the surface of the oxygenase domain, and is known to be phosphorylated by SP directed kinases in vivo. Without being bound by a single theory, it is possible that phosphorylation could be mediated by other kinases, or phosphorylation by MAP kinases could require additional input (e.g., prior phosphorylation of other sites), or phosphorylation by MAP kinases could require scaffolding components.

The tight spacing of control sites on the eNOS surface indicated a system in which eNOS is the junction of many signaling pathways and an active node in the resulting network. A simple example of multiple inputs is T497 phosphorylation, which interferes with CaM binding and hence activation. The sites associated with the AI are far more complex. Activating kinases such as PKA and AKT phosphorylate multiple targets in this region, which also includes the ERK/p38 binding site, the S602 ERK target, and the autoinhibitory helix, which locks the FMN binding domain to the NADPH binding domain. It is likely that phosphorylation of these sites affects binding of other regulators and phosphorylation of other sites. The complexity of the system may allow eNOS to behave more like an integrated circuit with multiple inputs and a nuanced array of outputs than a simple relay.

The inventive method and compositions demonstrated major control elements of nitric synthases, and signaling by several kinases. The invention encompasses identification of a MAP-kinase binding site on eNOS, identification of target sites of MAP kinase phosphorylation, and establishment of two molecular level mechanisms for NOS regulation by phosphorylation. Applications include novel negative feedback regulatory networks involving kinases (Akt, ERK2, and RSK), phosphatases, and eNOS in response to glucose, insulin, and other signals, with relevance to, e.g., diabetes and cardiac function.

A network model can be mathematically modeled using extensible software developed for this purpose (e.g., BioNetGen, freeware originally developed at Los Alamos). The invention implements new information about molecular states. Preliminary results in which systems of ordinary differential equations (ODE) describing the response of a partial network to perturbations (e.g., a VEGF signal) indicate that transient signals are generated downstream.

Cultured endothelial cells express eNOS and kinases in the associated network. The time course of signals (e.g., phosphorylated proteins, NO) in response to perturbations (e.g., extracellular signals such as bradykinin and VEGF) are evaluated by Western Blots and confocal microscopy. This information proves significance of in vivo results, informs the choice of modeling parameters, permits the design of reconstituted networks, and allows understanding of network behavior through modeling.

One embodiment is elucidation of interaction patterns underlying control of NO signaling and related signal transduction pathways, the dynamics of the effects of external signals such as Ca²⁺ influx, and the role of signaling molecules such as bradykinin, VEGF, insulin and glucose. These signal transduction networks are involved in the regulation of complex physiological processes including homeostatic mechanisms (e.g., vascular tone, insulin secretion) and adaptive and developmental mechanisms (e.g., angiogenesis). Since the identification of the major calmodulin displaced control element in NO synthases, there are new modes of regulation based on phosphorylation of specific residues by a growing roster of kinases (see table below for examples), by modifications that target eNOS to cellular compartments, and by interactions with proteins that result in the formation of functional or inhibitory complexes. Interaction of these inputs and the nature and behavior of the resulting network are disclosed.

Nitric oxide signaling is central to the control of physiological processes that are essential to normal function, pathophysiological conditions, and adaptation. Nitric oxide synthases (NOS) play multiple roles in such diverse physiological processes as control of vascular tone, signal transduction in the central nervous system, and immune response, and are significant in pathological conditions. NO generated by nNOS functions as a neurotransmitter. NO produced by eNOS regulates vascular tone, cardiac function, smooth muscle tension, and promotes angiogenesis.

Calcium/calmodulin (Ca^{2+/}CaM) is the primary regulator of signal generating NOS enzymes, but eNOS is imbedded in a complex signal transduction network including kinases, phosphatases, scaffolds, and inhibitors. The discovery of eNOS activation by kinases such as Akt and PKA via phosphorylation at S615, S633, and S1077 opened a new window on regulation of NO production. Human eNOS is phosphorylated at Y81, S114, T495, S615, S633, and S1177, corresponding to Y83, S116, T497, S617, S635, and S1179 in bovine eNOS; T95/97 and other sites may also be phosphorylated.

In the original model of Ca^{2÷} regulated NO signaling, an inward flux of Ca²⁺ (e.g., via shear activated channels) activates eNOS via CaM, and NO diffusing to soluble guanylate cyclase (sGC) produced the response (e.g., in smooth muscle cells this leads to relaxation). This mode of activation is primary, but numerous additional inputs serve essential functions. For example, adaption is essential during development, aging, and in response to other perturbations, and adjustment of NO production by signaling networks is one aspect. S615, S633, and S1077 phosphorylation enhance NO production, while T495 inhibits NO synthesis by preventing CaM binding to its adjacent canonical site. S600 is phosphorylated by ERK, leading to inhibition by restricting conformational mobility. S114 phosphorylation by ERK has also been reported, and unlike some other purported ERK targets, it represents an authentic MAPK motif; surprisingly, S114 phosphorylation by ERK was not detected in vitro.

Targeting dynamic aspects of components and pathways is required to understand cellular communication and control. Methods to follow the time course of signaling events can in turn be simulated using systems of differential equations or delay differential equations, e.g., studies of IκB-NFκB regulation and kinase cascades. While negative feedback loops may be described as ‘inherently stable’ in biology and medicine, this is far from the case. Negative feedback leads to unstable oscillations unless the system parameters meet stringent criteria; oscillatory behavior of this kind is a feature of many biological networks.

Control in the signal generating nitric oxide synthase isoforms eNOS (endothelial) and nNOS (neuronal) is centered in control elements associated with electron delivery by the reductase domains. The figures below show the location of key elements in the eNOS reductase domain. The CaM binding site of eNOS is located directly above the structures shown, and calcium stimulated CaM binding displaces the extended autoinhibitory element, allowing the enzyme to move through obligatory conformational intermediates. Ca²⁺ control allows direct rapid regulation by ion fluxes via the opening of stretch activated channels in the vascular system, Ca²⁺ release in neurons, and from the sarcoplasmic reticulum in muscle. Important secondary regulation takes place through protein-protein interaction and covalent modification. Of special importance are phosphorylation of residues such as T495, S633, S615, S600 and S1177 shown above, and others listed in the following Table 3, showing phosphorylation of specific eNOS residues by kinases and their agonists:

| Kinase | Site | Agonists |
|---|---|---|
| Akt | S1177, 615 | VEGF, IGF, shear stress, estrogen, |
| | | simvastatin |
| AMPK | S1177, T495 | [AMP]i |
| PKC | T495 | PMA |
| PKA | S1177, S633 | Iloprost |
| PKG | S1177, S633 | Atrial natriuretic peptide, guanylin, |
| | | etc. |
| JnK | S116 | Shear stress |
| MAP kinase | S602 | Bradykinin |

The placement of these residues in the structure suggests that modifications at some positions affect interactions at adjacent or overlapped positions. For example, the inventors have demonstrated that T495 phosphorylation prevents calmodulin from binding to eNOS; S633 phosphorylation likely inhibits MAP kinase binding to the site on the autoinhibitory element, and S600 and S615 phosphorylation may be mutually inhibitory. In the N terminal region, sites such as S56 or S58 may affect trafficking of eNOS to the membrane via myristoylation and palmitoylation at sites at G2, C15, and C 26.

Control element deletion mutants and phosphomimetic and phosphonull eNOS mutants that are available or are produced using site directed methods are used to investigate the roles of these sites in NO signaling. Phosphomimetic mutants have aspartate or gluatamate residues (D or E) substituted for serine, threonine or tyrosine (S, T or Y); introduction of a negatively charged residue usually mimics phosphorylation. Phosphonull mutants have alanine residues substituted for alcoholic amino acid phosphorylation targets, and these cannot be phosphorylated. Control element deletions have the AI and/or the C terminal extension deleted, producing enzymes with altered calmodulin control.

Such mutants permit study of interactions of eNOS with purified signal transduction components, including kinases and proteins such as calmodulin, caveolin, and HSP90. Binding is studied using Forte and Biacore biosensors, providing a quantitative measure of interactions between effectors on the surface of NOS. Enzymatic assays directly measure inhibition and activation, and fluorescence experiments reveal conformational effects by monitoring the interaction of FMN with other prosthetic groups in eNOS.

Mutants expressed in mammalian cell culture, together with specific inhibitors of signal transduction components, will allow perturbation of signal networks in cultured cells. For example, specify the resting state of the system using Western blots with phosphorylation specific antibodies, and observe the time course after introducing a signal (e.g., bradykinin or insulin); measuring NO production and using green fluorescence protein labeled components to observe trafficking in response to signaling.

eNOS and nNOS, but not iNOS, have autoinhibitory control elements (AE) with conserved and divergent regions. Midway through the AE, a single base insertion destroys amino acid conservation, although residual conservation at the DNA level is retained. This frame shift generates a pentabasic MAPK binding site in eNOS and the phosphorylation site at S633 two residues later. Immediately after this a second insertion restores frame, allowing amino acid homology to resume and producing a functional protein with altered control.

There are likely orders of magnitude more such binding sites in the human genome than can be accounted for by random chance or conventional homology. Paired frame shifts are likely a major factor in evolution of control and signaling, there are likely many examples of this in signaling proteins; the shifts rapidly generate high local diversity, which is then trapped by selection in favorable cases.

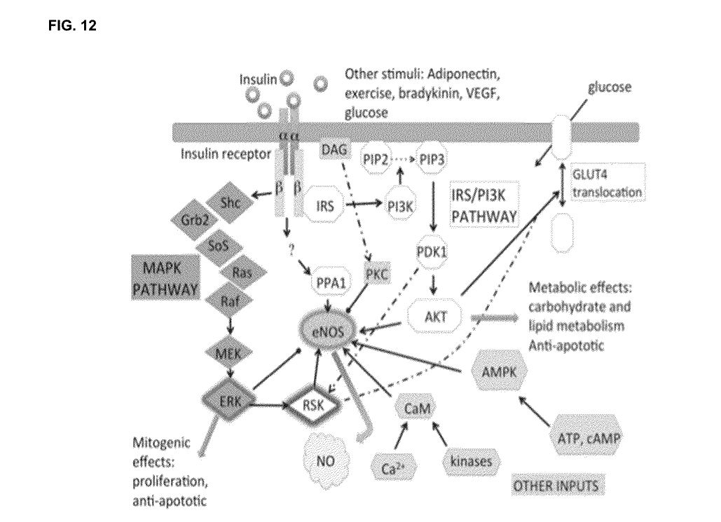

Integration of signaling pathways with eNOS, which serves as a signaling junction box (FIG. 12) receiving many inputs that are interpreted with great precision, creating a rheostat-like regulation of NO production. Similar network diagrams can be made for other receptors such as bradykinin and some components (e.g., the MAP kinase ERK, the kinases RSK, Akt and PKC) are also important nodes with multiple inputs and outputs. Off states are created in an environment devoid of calcium replete calmodulin and/or through phosphorylation at T495 (by PKC). T495 phosphorylation prevents calmodulin binding, creating an enzyme that cannot be activated by calmodulin. Other phosphorylation modifications exert varying degrees of control on NOS activity once calmodulin binds; some activate and some inhibit. Ser1177 phosphorylation activates eNOS (Table 2) and is a substrate for a number of enzymes with Akt the most studied. For at least one agonist, bradykinin, Akt is not activated in the time frame required for Ser1177 phosphorylation in the initial phase of activation. The other known activating sites are S615 and S633 (Table 2); phosphorylation at S633 has been shown to activate eNOS in the absence of calcium. Alteration of eNOS activity by MAP kinase has been controversial, with some suggesting activation and others inhibition. This is likely a function of different cell types and/or activators, making interpretations from a limited number of time points, attempting to attribute changes in a very dynamic system to one input, and failure to consider that MAP kinase activates downstream kinases including RSK which are likely NOS modulators.

eNOS phosphorylation sites for MAP kinases have not been previously described. The invention identifies three MAP kinase phosphorylation sites on bovine eNOS; two of these sites are conserved in human (S56, S600). Data also show that phosphorylation of eNOS by MAP kinase (ERK) is inhibitory. Understanding of regulation of this novel phosphorylation site and its interplay with cellar regulation of eNOS is demonstrated.

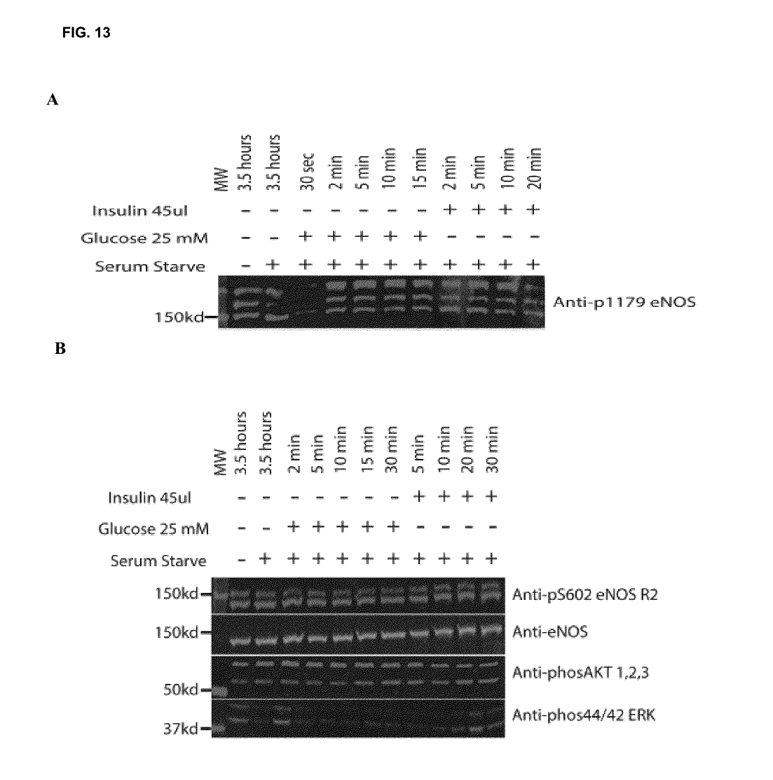

Phosphorylation of sites on eNOS and associated signaling components in cultured cells were done by Western blot analysis of samples taken at intervals after applying a stimulus with phosphospecific antibodies. FIG. 13 shows the phosphorylation of the novel antibody S602 and S1179 (eNOS is the bottom band for both blots) of eNOS in cultured endothelial cells; serum starvation had little immediate effect, but was required to observe the full effects of glucose or insulin treatment, particularly on the S1179 site. eNOS phosphorylation oscillates at p1179 and is altered by treatment with kinase inhibitors (data not shown). FIG. 13A shows lysates from BAEC cells harvested at 95% confluence after being serum starved for 3.5 hours and then treated with glucose or insulin for the indicated times. Equal amounts of lysate were probed for anti-pS1179. FIG. 13B is the same as FIG. 13A expect for timing and antibodies.

eNOS is quickly activated in response to inputs such as bradykinin, peaking in 1 to 3 minutes. Many studies, however, examine later time points or only one point. This provides an incomplete and misleading picture.

The inventors demonstrated oscillations in response to external signals (FIG. 13, pS1179 phosphorylation bottom band) for qualitative and quantitative understanding of how signals such as glucose and growth factors might impact the dynamics of these signaling changes. There may be oscillations of these modifications impacted by the presence of growth factors and/or glucose (FIG. 13).

Treatment of serum starved cells with insulin leads to modest changes in S1179 and S602 phosphorylation (FIG. 13) while as shown in FIG. 13A treatment of serum starved cells with a bolus of glucose produces nearly complete loss of S1179 phosphorylation within 30 seconds. S1179 phosphorylation recovers within two minutes to more than half its initial level, and then falls again, recovering to approximately half its initial value. FIG. 13A also shows the effect of glucose treatment on S602 phosphorylation. The dynamic range of the S602 phosphorylation is much smaller than that of S1179; for S602, phosphorylation rises moderately and then returns.

There is direct and indirect evidence of RSK interaction with eNOS for regulation. In vitro RSK phosphorylates eNOS at S1177, resulting in increased NO production, similar to Akt phosphorylation in vitro (data not shown). RSK has little apparent impact on bradykinin induced phosphorylation of eNOS (data not shown). Blotting with antibody to unphosphorylated eNOS confirms the assignment of the band to phosphorylated eNOS (2^{nd} panel in FIG. 13B). Phosphospecific antibodies react poorly with purified unphosphorylated eNOS, but in some cases cross-react with several other proteins of different molecular weights, likely because these proteins contain a similar motif and are phosphorylated by the same family of kinases.

Cell-based and in vitro assays are used to better understand the role of MAP kinase and RSK in eNOS activation. Endogenous eNOS and transfected eNOS variants determine changes in phosphorylation status and/or activity; data and information will inform creation of a computational network literature capable of predicting novel signaling connections to eNOS.

eNOS as a substrate for PKC in the presence of CAM is being evaluated, with the following possible outcomes: eNOS-CAM might not be a substrate since CAM binds tightly and may block access to T495, or eNOS-CAM could be a substrate, leading to phosphorylation and CAM displacement. Results are focused on mechanisms of control. If the complex is not a substrate, T495 phosphorylation likely controls eNOS by preventing activation rather than reversing it, but if PKC phosphorylates the complex it would suggest that PKC has two mechanisms of regulation prevention of activation and the ability to feedback inhibit. Treatment with the MEK inhibitor PD 98059 does not block the dissociation of ERK from eNOS complexes. This suggests an additional factor in MAP kinase activation that mediates the removal of ERK from the signaling complex.

Assaying purified eNOS and observing structural changes using fluorescence analysis provides unique tests of how individual changes impact eNOS function. The nuanced function of eNOS, interpreting varied signals to determine NO output, makes it ripe for computational analysis. In creating this network, the inventors discovered a new node of signaling impacting NO production by eNOS.

FIG. 9C, top panel, is an example of in situ results using the anti-pS602 antibody. FIG. 13A shows comparable results from a commercially available pS1179 antibody. The bottom band of the triplet is actual eNOS reaction with pS1179; the lower band of the doublet in FIG. 13B is pS602 detection of phosphorylated eNOS, it appears that pS602 is at least as good as the commercially available reagent.

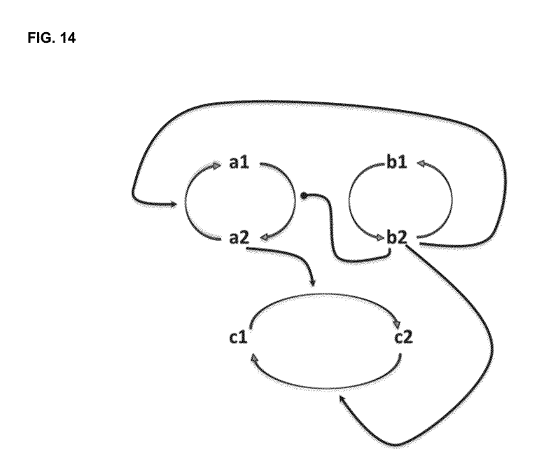

For computational modeling of signal transduction pathways, oscillatory behavior is intimately connected with feedback mechanisms. FIG. 14 shows a simple feedback model capable of reproducing the oscillations observed in FIGS. 12 and 13. A and B represent species that can be activated by phosphorylation; in the simplest case A would represent a kinase and B a phosphatase, but more generally A and B can represent sequences that contain several components. In the simple case A1 and A2 represent respectively the inactive form of A and the active, phosphorylated form of A, and in the same way B1 and B2 represent the inactive and active forms of B. For simplicity, only a single phosphorylation site is considered.

In this model A2 but not A1 phosphorylates and activates B1, forming B2. B2 dephosphorylates/inactivates A2 and/or inactivates the enzyme that phosphorylates AI; both mechanisms produce negative feedback. The coupled system can be represented by the system of differential equations:

$$\frac{\partial b 1}{\partial t} = \frac{- b 1}{{K}_{bn} + b 1} {K}_{b 1} - \frac{b 1}{{K}_{bm} + b 1} {K}_{b 2} a 2 + \frac{b 2}{{K}_{bo} + b 2} {K}_{b 3}$$

$$\frac{\partial b 2}{\partial t} = \frac{- \partial b 1}{\partial t}$$

$$\frac{\partial a 1}{\partial t} = \frac{- a 1}{{K}_{an} + a 1} {K}_{a 1} f ( b 2 ) - \frac{a 1}{{K}_{am} + a 1} {K}_{a 2} g ( b 2 ) + \frac{a 2}{{K}_{ao} + a 2} {K}_{a 3} b 2 + \frac{a 2}{{K}_{ap} + a 2} {K}_{a 4}$$

$$\frac{\partial a 2}{\partial t} = \frac{- \partial a 1}{\partial t}$$

A secondary feature of the model is provided by functions that represent the steady state partition of the kinase that converts a1 to a2. These functions are:

f(b2)=r1/(b2+r1+r2) and g(b2)=(b2+r2)/(b2+r1+r2)

Here Kₐ₁, Kₐ₂, Kₐ₃, Kₐ₄, K_{b1}, K_{b2}, and K_{b} are rate constants for interconversion of the forms of A and B, and a1, a2, b1, and b2 represent concentrations of A1, A2, B1, and B2. Parameters of the form Kₐₘ through Kₐₚ and K_{bm} through K_{bo} are effective Michaelis constants for enzymes interconverting these species, and r1 and r2 are ratios of rate constants for kinase activation and deactivation.

Component C is phosphorylated by A2 and dephosphorylated by B2; its state is described by the equations

$$\frac{\partial c 1}{\partial t} = \frac{( - c 1 )}{( {K}_{cn} + c 1 )} {K}_{c 1} - \frac{c 1}{( {K}_{cm} + c 1 )} {K}_{c 2} a 2 + \frac{c 2}{( {K}_{co} + c 2 )} {K}_{c 3} b 2 + \frac{c 2}{( {K}_{cp} + c 2 )} {K}_{c 4}$$

$$\frac{\partial c 2}{\partial t} = \frac{- \partial c 1}{\partial t}$$

As written, the a and b components are functions of each other but not of c components, so there is no feedback from C to either of them; the phosphorylation state of C is an integrated output of a2 and b2. In these equations a1, a2, b1, b2, c1, and c2 are functions of time. Written as a1 (t), a2(t) etc. with no delays, the model generates roughly exponential trajectories in time for the components of A and B, with oscillatory behavior limited to slight overshoots (not shown). The differential equations can be written in delay form, introducing delays τₐ, τ_{ab}, and τ_{b} into the terms connecting A and B components. Delays of this form could be introduced by obligatory formation and decay of protein complexes, diffusion, mobilization to other compartments, and participation of unspecified intermediates. In delay form, the equations can be written

$$\frac{\partial b 1}{\partial t} = \frac{- b 1}{{K}_{bn} + b 1} {K}_{b 1} - \frac{b 1}{{K}_{bm} + b 1} {K}_{b 2} a 2 ( t - {τ}_{a} ) + \frac{b 2}{{K}_{bo} + b 2} {K}_{b 3}$$

$$\frac{\partial a 1}{\partial t} = \frac{- a 1}{{K}_{an} + a 1} {K}_{a 1} f ( b 2 ( t - {τ}_{c} ) ) - \frac{a 1}{{K}_{am} + a 1} {K}_{a 2} g ( b 2 ( t - {τ}_{c} ) ) + \frac{a 2}{{K}_{ao} + a 2} {K}_{a 3} b 2 ( t - {τ}_{b} ) + \frac{a 2}{{K}_{ap} + a 2} {K}_{a 4}$$

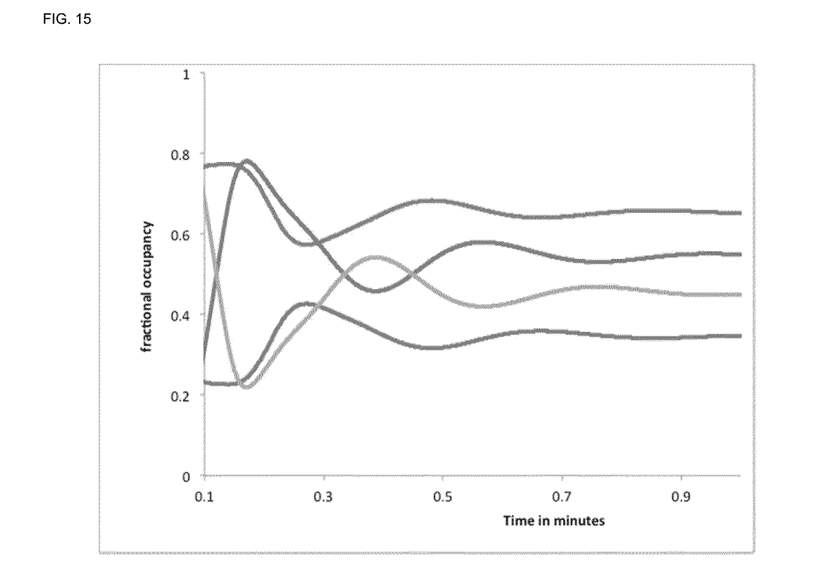

Terms in a1, b1, a2 and b2 that do not have delays should be understood as functions of undelayed time; e.g., a2(t) rather than a2(t−τₐ). Simulation of the experimental data was undertaken using fourth order Runge-Kutta integration of the system of delay differential equations. For delays comparable to the inverse of the rates, oscillations were generated closely matching the observed results. FIG. 15 shows a simulation of a1/a2 and b1/b2 as a function of time. No direct data are available for the phosphatase arm identified with b2, but the trajectory of the phosphorylation state of Akt after glucose treatment closely resembles that of a2, the uppermost trace.

The corresponding simulation of the time dependence of c2 closely matches the phosphorylation of S1177 on eNOS. Although phosphatase activation has not yet been observed directly, the oscillations of Akt and eNOS phosphorylation state could not be generated without participation of such a component. The oscillatory behavior is strongly indicative of a feedback loop

Mathematical modeling of cellular signaling pathways is a way to integrate information produced in experimental studies. Mathematical descriptions of certain processes, such as enzyme kinetics, have helped to establish the role of feedback and feed-forward mechanisms in complex biological phenomena. A mathematical analysis is being conducted by two approaches.

One approach extends the current quantitative computational modeling. This primarily entails the analysis by numerical simulation of a system of ordinary differential equations (ODEs) or delay differential equations (DDEs). A trial, small system of DDEs governing the kinase and phosphatase processes has produced an oscillatory phosphorylation-dephosphorylation behavior similar to that observed experimentally. This system will be supplemented with equations governing additional species, and simulations will use open source software and mathematical packages such as MATLAB. An advantage to quantitative modeling is the flexibility allowed by considering as large a system of equations as is considered relevant. Such a model can be presented as a general template that allows for adaptation as understanding of the processes develops. Experimental results are integrated at various stages as parameters are determined for a proposed model for integration of mathematical and experimental modeling. The availability of robust software facilitates numerical simulations; however, large systems of equations rarely lend themselves to subtle analytical techniques.

The second approach is qualitative and studies reduced systems of equations derived via quasi-steady state analysis (e.g. considering “relatively slow” processes as constant). Stability and bifurcation analyses can illuminate parameter interactions that govern observed phenomena. In particular, it may be possible to isolate specific parameter relationships that serve as a critical threshold for types of pathway dynamics, e.g., between decay and oscillatory behaviors. Identification of such parameter relationships bolster conclusions drawn from experimental observations, and qualitative analysis may inform experimental design by suggesting signaling pathway interactions.

A new signaling network involving important signal transduction elements is disclosed. These include MAP kinases, RSK, and endothelial nitric oxide synthase, the primary regulator of human vascular tone and hence blood pressure and an important signal generator in cardiac function, development of new vessels, i.e., angiogenesis, insulin secretion, and sexual function. The development of new algorithms, generation of tools with potential to discover many evolutionary adaptations in systems that span much of biology are feasible. Coalignment tools will find broad application for laboratories performing site directed mutagenesis. Full scale dynamic simulations, previously relatively rare, and mathematical underpinnings of transient signals and oscillations provide more flexible treatments capable of handling detailed networks with arrays of interaction parameters.

Applicants incorporate by reference the material contained in the accompanying computer readable Sequence Listing identified as Sequence_Listing_ST25.txt, having a file creation date of Feb. 15, 2013, 11:33 a.m., and file size of 10.1 kilobytes.

Each of the following are expressly incorporated by reference herein in its entirety:
1. Cargnello and Roux (2011) Activation and function of the MAPKs and their substrates, the MAPK-activated protein kinases, Microbiol. Mol. Biol. Rev. 75, 50-83.
2. Cuadrado and Nebreda (2010) Mechanisms and functions of p38 MAPK signaling, Biochem. J. 429, 403-417.
3. Zarubin and Han (2005) Activation and signaling of the p38 MAP kinase pathway, Cell Res. 15, 11-18.
4. Bardwell et al. (2009) Selectivity of docking sites in MAPK kinases, J. Biol. Chem. 284, 13165-13173.
5. Gaestel (2008) Specificity of signaling from MAPKs to MAPKAPKs: kinases' tango nuevo, Front. Biosci. 13, 6050-6059.
6. Ignarro et al. (1987) Endothelium-derived relaxing factor produced and released from artery and vein is nitric oxide, Proc. Natl. Acad. Sci. 84, 9265-9269.
7. Furchgott (1988) Studies in relaxation of rabbit aorta by sodium nitrite: the basis for the proposal that the acid activatible factor from bovine retractor penis is inorganic nitrite, in Vasodilation: Vascular Smooth Muscle Peptides, Autonomic Nerves and Endothelium, pp 401-404, Raven Press, New York.
8. Papapetropoulos et al. (1997) Nitric oxide production contributes to the angiogenic properties of vascular endothelial growth factor in human endothelial cells, J. Clin. Invest. 100, 3131-3139.
9. Corbett et al. (1993) Nitric oxide mediates cytokine-induced inhibition of insulin secretion by human islets of Langerhans, Proc. Natl. Acad. Sci. 90, 1731-1735.
10. Henningsson et al. (2002) Role of nitric oxide synthase isoforms in glucose-stimulated insulin release, Am. J. Physiol. 283, C296-304.
11. Bernier et al. (2000) Bradykinin-regulated Interactions of the Mitogen-activated Protein Kinase Pathway with the Endothelial Nitric-oxide Synthase, J Biol. Chem. 275, 30707-30715
12. Roux and Blenis (2004) ERK and p38 MAPK-Activated Protein Kinases: a Family of Protein Kinases with Diverse Biological Functions, Microbiol. Mol. Biol. Rev. 68, 320-344
13. Enslena and Davis (2001) Regulation of MAP kinases by docking domains, Biology of the Cell 93, 5-14.
14. Sharrocks et al. (2000) Docking domains and substrate specificity determination for MAP kinases, TIBS 25, 448-453.
15. Bardwell et al. (2009) Selectivity of Docking Sites in MAPK Kinases J. Biol. Chem. 284, 13165-13173.
16. Salerno et al. (1997) An autoinhibitory control element defines calcium-regulated isoforms of nitric oxide synthase, J. Biol. Chem. 272, 29769-29777.
17. Boo et al. (2006) Coordinated regulation of endothelial nitric oxide synthase activity by phosphorylation and subcellular localization, Free Rad. Biol. & Med. 41, 144-153.
18. Mount et al. (2007) Regulation of endothelial and myocardial NO synthesis by multi-site eNOS phosphorylation, J. Mol. Cell. Cardiology 42, 271-279.
19. Fulton et al. (2001) Post-translational control of endothelial nitric oxide synthase: why isn't calcium/calmodulin enough? J. Pharmacology And Experimental Therapeutics 299, 818-824.
20. Garcin et al. (2004) Structural basis for isozyme-specific regulation of electron transfer in nitric-oxide synthase, J. Biol. Chem. 279, 37918-37927.
21. McMurry et al. (2011) Rate, affinity and calcium dependence of nitric oxide synthase isoform binding to the primary physiological regulator calmodulin. FEBS J. 278, 4943-54.
22. Gerber and Ortiz de Montellano (1995) Neuronal nitric oxide synthase. Expression in Escherichia coli, irreversible inhibition by phenyldiazene, and active site topology, J. Biol. Chem. 270, 17791-17796.
23. Roman et al. (1995) High-level expression of functional rat neuronal nitric oxide synthase in Escherichia coli, Proc. Natl. Acad. Sci. 92, 8428-8432.
24. Martasek et al. (1996) Characterization of bovine endothelial nitric oxide synthase expressed in E. coli, Biochem. Biophys. Res. Corn. 219, 359-365.
25. Gross (1996) Microtiter plate assay for determining kinetics of nitric oxide synthesis, Methods Enz. 268, 159-168.
26. Newman (2004) Differential activation of nitric-oxide synthase isozymes by calmodulin-troponin C chimeras, J. Biol. Chem. 279, 33547-33557.
26. Chrestensen (2004) MAPKAP kinase 2 phosphorylates tristetraprolin on in vivo sites including Ser178, a site required for 14-3-3 binding, J. Biol. Chem. 279, 10176-10184.
27. Abdiche (2008) Determining kinetics and affinities of protein interactions using a parallel real-time label-free biosensor, the Octet, Anal. Biochem. 377, 209-217.
28. Roman and Masters (2006) Electron transfer by neuronal nitric-oxide synthase is regulated by concerted interaction of calmodulin and two intrinsic regulatory elements J. Biol. Chem. 281, 23111-23118.
29. Lukas et al. (2004) Catalysis and function of the p38 alpha MK2a signaling complex, Biochem. 43, 9950-9960.
30. Kumar and Sudhakaran (2009) Negative modulation of eNOS by laminin involving post-translational phosphorylation. J Cell Physiol. 219(1)123-31.
31. Kan et al. (2008) p38 MAPK-dependent eNOS upregulation is critical for 17beta-estradiol-mediated cardioprotection following trauma-hemorrhage. Am J Physiol Heart Circ Physiol. 294, H2627-36.
32. Gao et al. (2004) Docking of endothelial nitric oxide synthase (eNOS) to the mitochondrial outer membrane: a pentabasic amino acid sequence in the autoinhibitory domain of eNOS targets a proteinase K-cleavable peptide on the cytoplasmic face of mitochondria, J. Biol. Chem. 279, 15968-15974.

The embodiments shown and described in the specification are only specific embodiments of inventors who are skilled in the art and are not limiting in any way. Therefore, various changes, modifications, or alterations to those embodiments may be made without departing from the spirit of the invention in the scope of the following claims. The references cited are expressly incorporated by reference herein in their entirety.

What is claimed is:

1. An antibody that selectively binds to a phosphorylated form of endothelial nitric oxide synthase (eNOS) and does not bind to a non-phosphorylated form of eNOS, where the antibody selectively binds to pS602 in bovine eNOS, pS600 in human eNOS; pT46 in bovine eNOS, pT44 in human eNOS; pS58 in bovine eNOS, pS56 in human eNOS; or pS116 in bovine eNOS, pS114 in human eNOS.

2. The antibody of claim 1 where eNOS is phosphorylated by a mitogen activated protein (MAP) kinase.

3. The antibody of claim 2 where the MAP kinase is selected from the group consisting of ERK, p38, and JNK.

4. The antibody of claim 1 selected from the group consisting of a polyclonal antibody, a monoclonal antibody, a chimeric antibody, a single chain antibody, a humanized antibody, a Fab fragment, a F(ab′)₂ fragment, and fragments produced by a Fab expression library.

5. The antibody of claim 1 labeled with a detectable marker selected from the group consisting of a chemiluminescent moiety, an enzymatic moiety, a fluorescent moiety, a radioactive moiety, and combinations thereof.

6. A method for determining if a biological sample contains a phosphorylated form of endothelial nitric oxide synthase (eNOS), the method comprising
contacting the biological sample with an antibody that selectively binds to a phosphorylated form of endothelial nitric oxide synthase (eNOS) and does not bind to a non-phosphorylated form of eNOS; and
determining whether the antibody binds to the biological sample,
where binding of the antibody to the biological sample indicates that the biological sample contains the phosphorylated form of eNOS.

7. The method of claim 6 where the endothelial nitric oxide synthase is phosphorylated by a mitogen activated protein (MAP) kinase.

8. The method of claim 7 where the MAP kinase is selected from the group consisting of ERK, p38, and JNK.

9. The method of claim 6 where the antibody selectively binds to pS602 in bovine eNOS, pS600 in human eNOS; pT46 in bovine eNOS, pT44 in human eNOS; pS58 in bovine eNOS, pS56 in human eNOS; or pS116 in bovine eNOS, pS114 in human eNOS.

10. The method of claim 6 where the antibody is labeled with a detectable marker selected from the group consisting of a chemiluminescent moiety, an enzymatic moiety, a fluorescent moiety, a radioactive moiety, and combinations thereof.

11. The method of claim 6 where binding is determined by a method selected from the group consisting of Western blotting and immunohistochemical staining.

12. The method of claim 6 further comprising screening a compound for effectiveness in phosphorylating eNOS by combining the compound with a non-phosphorylated eNOS to form a mixture under conditions suitable for the compound to phosphorylate eNOS; then contacting the mixture with an antibody that selectively binds to a phosphorylated form of eNOS but not to a non-phosphorylated form of eNOS; and
detecting eNOS phosphorylation in the mixture where eNOS phosphorylation indicates that the compound phosphorylates and/or causes phosphorylation of eNOS.

13. The method of claim 12 where the antibody selectively binds to pS602 in bovine eNOS, pS600 in human eNOS; pT46 in bovine eNOS, pT44 in human eNOS; pS58 in bovine eNOS, pS56 in human eNOS; or pS116 in bovine eNOS, pS114 in human eNOS.

14. The method of claim 12 where the compound and/or the non-phosphorylated NOS are present in a biological sample.

15. The method of claim 14 where the biological sample is a cell lysate.

16. The method of claim 12 where the antibody is labeled with a detectable marker selected from the group consisting of a chemiluminescent moiety, an enzymatic moiety, a fluorescent moiety, a radioactive moiety, and combinations thereof.

17. The method of claim 12 where detecting is by a method selected from the group consisting of Western blotting and immunohistochemical staining.

18. An anti-mammalian pS602 endothelial nitric oxide synthase antibody.

19. A kit comprising the antibody of claim 1 and instructions for using the antibody to detect phosphorylated endothelial nitric oxide synthase.

20. A kit comprising the antibody of claim 18 and instructions for using the antibody to detect phosphorylated endothelial nitric oxide synthase.


SEQUENCE LISTING (25 sequences)

SEQ ID NO: 1 - protein, 29 aa, Artificial Sequence
  rat nNOS
NSVSCSDPLVSSWRRKRKESSNTDSAGAL

SEQ ID NO: 2 - protein, 29 aa, Artificial Sequence
  bovine eNOS
NSVSCSDPLVSSWRRKRKESSNTDSAGAL

SEQ ID NO: 3 - protein, 29 aa, Artificial Sequence
  Human MK2-B
IKKIEDASNPLLLKRRKKARALEAAALAH

SEQ ID NO: 4 - protein, 29 aa, Artificial Sequence
  Human MK3
IKDLKTSNNRLLNKRRKKQAGSSSASQGC

SEQ ID NO: 5 - protein, 20 aa, Artificial Sequence
  Human MKK6
MSQSKGKKRNPGLKIPKEAF

SEQ ID NO: 6 - DNA, 166 bp, Artificial Sequence
  Human nNOS
tgaggcaccccaactctgtgcaggaagaaaggaagagctacaaggtccgattcaacagcgtctcctcctactctgactcccaaaaatcatcaggcgatgggcccgacctcagagacaactttgagagtgctggacccctggccaatgtgaggttctcagtttttgg

SEQ ID NO: 7 - DNA, 166 bp, Artificial Sequence
  rat nNOS
tgaggcaccccaactctgtgcaggaggagagaaagagctacaaggtccgattcaacagcgtctcctcctattctgactcccgaaagtcatcgggcgacggacccgacctcagagacaactttgaaagtactggacccctggccaatgtgaggttctcagtgttcgg

SEQ ID NO: 8 - DNA, 175 bp, Artificial Sequence
  Human eNOS
tgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgg

SEQ ID NO: 9 - DNA, 175 bp, Artificial Sequence
  bovine eNOS
tgtcggggccctacaacagctccccgcggccggaacagcacaagagttacaagatccgcttcaacagcgtctcctgctcagacccgctggtgtcctcctggcggcggaagagaaaggagtccagcaacacagacagtgcgggggccctggggaccctcaggttctgtgtgttcgg

SEQ ID NO: 10 - DNA, 40 bp, Artificial Sequence
  Human iNOS
tgaaagagctcaacaacaaattcaggtacgctgtgtttgg

SEQ ID NO: 11 - protein, 55 aa, Artificial Sequence
  Human nNOS
RHPNSVQEERKSYKVRFNSVSSYSDSQKSSGDGPDLRDNFESAGPLANVRFSVFG

SEQ ID NO: 12 - protein, 55 aa, Artificial Sequence
  rat nNOS
RHPNSVQEERKSYSVRFNSVSSYSDSRKSSGDGPDLRDNFESTGPLANVRFSVFG

SEQ ID NO: 13 - protein, 58 aa, Artificial Sequence
  Human eNOS
SGPYNSSPRPEQHKSYKIRFNSISCSDPLVSSWRRKRKESSNTDSAGALGTLRFCVFG

SEQ ID NO: 14 - protein, 58 aa, Artificial Sequence
  Bovine eNOS
SGPYNSSPRPEQHKSYKIRFNSVSCSDPLVSSWRRKRKESSNTDSAGALGTLRFCVFG

SEQ ID NO: 15 - protein, 13 aa, Artificial Sequence
  Human iNOS
KELNNKFRYAVFG

SEQ ID NO: 16 - protein, 31 aa, Artificial Sequence
  eNOS tryptic digests
RAPAPATPHAPDHSPAPNSPTLTRPPEGPKF

SEQ ID NO: 17 - protein, 31 aa, Artificial Sequence; identical to SEQ ID NO: 16
  eNOS tryptic digests

SEQ ID NO: 18 - protein, 31 aa, Artificial Sequence; identical to SEQ ID NO: 16
  eNOS tryptic digests

SEQ ID NO: 19 - protein, 34 aa, Artificial Sequence
  eNOS tryptic digests
RAPAPATPHAPDHSPAPNSPTLTRPPEGPKFPRV

SEQ ID NO: 20 - protein, 31 aa, Artificial Sequence; identical to SEQ ID NO: 16
  eNOS tryptic digests

SEQ ID NO: 21 - protein, 23 aa, Artificial Sequence
  eNOS tryptic digests
FAAALMEMSGPYNSSPRPEQHKS

SEQ ID NO: 22 - protein, 21 aa, Artificial Sequence
  eNOS tryptic digests
AALMEMSGPYNSSPRPEQHKS

SEQ ID NO: 23 - protein, 21 aa, Artificial Sequence
  eNOS tryptic digests
AALMEMSGPYNSSPRPEQHKS

SEQ ID NO: 24 - protein, 19 aa, Artificial Sequence
  eNOS tryptic digests
LMEMSGPYNSSPRPEQHKS

SEQ ID NO: 25 - protein, 26 aa, Artificial Sequence
  eNOS tryptic digests
ATPHAPDHSPAPNSPTLTRPPEGPKF